(19) United States
(12) Patent Application Publication
(10) Pub. No.: US 2003/0175242 A1
(43) Pub. Date: Sep. 18, 2003

(54) Cell therapy system

(76) Inventor: Micheal Gruenberg, Poway, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/246,293

(22) Filed: Sep. 17, 2002

Related U.S. Application Data
(60) Provisional application No. 60/322,626, filed on Sep. 17, 2001.

(51) Int. Cl. A61K 48/00; G06F 17/60

(52) U.S. Cl. 424/93.2; 705/2

(57) ABSTRACT

Systems and methods for manufacturing and distributing somatic cell therapy and gene therapy products are provided. The systems and methods include establishing a central processing facility and a plurality of satellite facilities administered under a single government license for conducting somatic cell or gene therapy, collecting source material at one of the satellite facilities from a first subject, transporting the source material from the first subject and delivering the source material to the central processing facility, processing the source material from the first subject at the central processing facility to produce a therapy product for administration to the same first subject, transporting the therapy product back to the satellite facility and administering the therapy product to the same first subject. All steps are performed under the control of the manufacturer.

### RELATED APPLICATIONS

Priority is claimed to U.S. provisional application Serial No. 60/322,626, filed Sep. 17, 2001, entitled “CELL THERAPY SYSTEM.” This application is related to U.S. application Ser. No. 08/506,668, converted to U.S. provisional application Serial No. 60/044,693, now abandoned; pending U.S. applications Ser. Nos. 08/700,565, 09/127,411, 09/127,142, 09/127,138, 09/127,141, 09/824,906, and International PCT application No. WO 97/05239. This application is also related to U.S. application Ser. No. 10/071,016, filed Feb. 7, 2002, to Micheal Gruenberg, entitled “Th1 Adoptive Immunotherapy,” and to U.S. application Ser. No. 09/957,194, filed Sep. 19, 2001, to Micheal Gruenberg, entitled “Th1 Adoptive Immunotherapy.” This application is also related to U.S. application Ser. No. 10/094,667, filed Mar. 7, 2002, to Micheal Gruenberg, entitled “RE-ACTIVATED T-CELLS FOR ADOPTIVE IMMUNOTHERAPY”, and to International PCT application Nos. PCT/US02/xxxx (attorney Docket No. 24731-504PC) and PCT/US02/xxxx (attorney Docket No. 24731-508PC), each filed the same day herewith. The subject matter of each of the provisional, utility and international applications is incorporated by reference in its entirety.

### TECHNICAL FIELD

Systems and methods for manufacturing and distributing somatic cell therapy and gene therapy products are provided. The systems and methods provided herein permits a manufacturer or other user to produce somatic cell therapy and gene therapy products such that the materials are under control of the manufacturer or user for the entire process. As a result, the systems and methods that comply with regularatory agency requirements, such as good manufacturing practice (CGMP) regulations for devices, biologics and drugs under 21 C.F.R. parts 211, 606 and 820 as applicable.

### BACKGROUND

Due to advances in scientific and technical knowledge over the past two decades, a new treatment modality has emerged that involves (1) removal of biological material from an individual or animal, (2) manipulation of the biological material in the laboratory, and (3) return of the biological material to the individual or animal as part of a therapeutic regimen. The biological material can contain living and/or non-living cells in whole or in part as the active ingredient of the biological material. This type of therapeutic regimen is known by many names, including “adoptive cell therapy” or “cellular immunotherapy” or “cancer vaccine” or “gene therapy”.

Examples of these types of treatments include the use of lymphokine activated killer (LAK) cells (see U.S. Pat. No. 4,690,915 issued to Rosenberg), tumor infiltrating lymphocytes (TIL) cells (see U.S. Pat. No. 5,126,132 issued to Rosenberg), cytotoxic T-cells (see U.S. Pat. No. 6,255,073 issued to Cai, et al.; U.S. Pat. No. 5,846,827 issued to Celis, et al.), expanded tumor draining lymph node cells (see U.S. Pat. No. 6,251,385 issued to Terman), genetically transformed stem cells (see U.S. Pat. No. 6,225,044 issued to Klein, et al.), mononuclear phagocytes (see U.S. Pat. No. 6,210,963 issued to Haddada, et al.), lymphocytes (see U.S. Pat. No. 6,194,207 issued to Bell, et al.; U.S. Pat. No. 5,443,983 issued to Ochoa, et al.; U.S. Pat. No 6,040,177 issued to Riddell, et al.; U.S. Pat. No. 5,766,920 issued to Babbitt, et al.), dendritic cells (see U.S. Pat. No. 6,210,662 issued to Laus, et al.), lymphocytes treated with oxidizing agents (see U.S. Pat. No. 6,204,058 issued to Bolton), and cellular vaccines (see U.S. Pat. No. 6,227,368 issued to Hiserodt, et al).

The U.S. Food and Drug Administration (FDA) refers to these therapies as “Somatic Cell and Gene Therapies”. As defined by the FDA, a “somatic cell therapy product” can be one or more autologous (self), allogeneic (intra-species), or xenogeneic (inter-species) cell(s) that have been propagated, expanded, selected, pharmacologically treated, or otherwise altered in biological characteristics ex-vivo to be administered to humans and applicable to the prevention, treatment, cure, diagnosis, or mitigation of disease or injuries. A “gene therapy product”, as defined by the FDA, can be one or more products that contain genetic material which are administered to modify and/or manipulate expression of genetic material and/or to alter biological properties of living cells.

The FDA issued a notice in the Federal Register of Oct. 14, 1993 (58 FR 53248), entitled “Application of Current Statutory Authorities to Human Somatic Cell Therapy Products and Gene Therapy Products”, and, later expanded the notice in the Can 28, 1996 Federal Register (61 FR 26523) and the Feb. 28, 1997 Federal Register (62 FR 9721). The notice provides the regulatory framework for manufacturing, testing and eventual marketing of a somatic cell therapy and/or a gene therapy product. In addition, the FDA considers a somatic cell therapy and/or a gene therapy product used as part of a therapeutic regimen to be a “drug” as that term is defined under 21 United States Code (USC) 321(g).

As a result, in the United States, manufacture of the somatic cell therapy and/or gene therapy product requires one or more government licenses. To obtain a government license, the manufacturer must prove that the therapy is safe and effective for its intended purpose in controlled clinical trials conducted under an Investigational New Drug (IND) application and the somatic cell therapy and/or gene therapy product must be produced in compliance with good manufacturing practice (CGMP) regulations for devices, biologics and drug, as applicable and as defined under 21 Code of Federal Regulations (CFR) parts 211, 606 and 820. The government license obtained by the manufacturer typically covers any facility that is owned and/or controlled by the manufacturer and that is used during a manufacturing process of the therapy product. Furthermore, each facility that is involved in the production of the therapy products must be licensed and approved by the FDA.

As an example of a CGMP regulatory requirements, the FDA requires that a manufacturer control (1) collection of biological material from a subject such as a human in need of therapy (i.e. a patient), (2) processing of the biological material into the somatic cell therapy or gene therapy product, and (3) administration of the somatic cell therapy or gene therapy product into a subject. More specifically, CGMP regulations require the manufacturer to control the (1) facilities, (2) personnel, (3) equipment, (4) documentation, and (5) procedures used during each step involved in manufacturing, distributing and administration of somatic cell and gene therapies. The manufacturer demonstrates compliance with CGMP by providing the FDA with “documented evidence which provides a high degree of assurance that a specific process will consistently produce a product meeting its pre-determined specifications and quality attributes.”

Consequently, manufacturers in order to meet FDA requirements under CGMP must (1) provide comprehensive documentation and documentation control methods, (2) validate collection, processing, shipping, and product administration procedures, and (3) develop raw material, in-process and final release specifications, methods, procedures and release criteria. As an example, documentation typically includes standard operating procedures (SOP), protocols, master production records, and/or log books that are maintained in the manufacturing facility to provide a chronological record of all equipment-related activities, such as equipment operation, room usage, environmental conditions and cleaning.

In addition, the manufacturer must implement a numbering system to identify and track any item in the manufacturing facility that will become a part of, or come in direct contact with, a somatic cell therapy and/or gene therapy product during the manufacturing process when following CGMP regulations. As a result, development of somatic cell therapy or gene therapy products that comply with FDA-mandated CGMP regulations is an arduous, complex, time-consuming and expensive task. Since there are currently no somatic or gene therapy products approved by the FDA, there are currently no examples of how to produce these therapies in a manner compliant with FDA regulations.

Several manufacturers have developed methods to produce somatic cell therapy and gene therapy products for clinical testing. These products are all in early phases of clinical development. Available production methods fall under two broad categories: (1) a central processing method, or (2) an on-site processing method. Neither of these methods comply with CGMP regulations as codified.

Since compliance with CGMP regulations is not strictly enforced by the FDA during early clinical testing of these products, if any of these products demonstrate safety and effectiveness in early clinical trials, new methods will be required in order to proceed into later phases of clinical testing and ultimatly to obtain FDA approval for marketing. When using a central processing method to manufacture and distribute somatic cell therapy and gene therapy products, arrangements are made to collect a subject's biological source material in an originating facility near the subject's location. Typically, the originating facility is an academic center, clinical laboratory, blood bank, hospital facility (e.g., outsubject, inpatient or surgical suite), physician's office or other healthcare facility. Next, the source material is shipped to a central processing facility, generally owned or controlled by the manufacturer in order to process and/or formulate the source material into a drug product. After processing and/or formulating, the drug product produced at the central processing facility is packaged and shipped back to the subject's location originating facility for administration to the subject.

Manufacturers that use the central processing method only have control of the portion of the process that occurs at the central processing facility. These manufacturers do not have control over the collection of the source material at the originating facility. These manufacturers also are not able to control how the product is stored, how long and under what conditions it is held and the method or manner in which it is administered to the subject. Significantly, in the case of autologous somatic cell and gene therapy products, it is not possible for manufacturers using the central processing method to assure that the personnel at the originating facility administered the product to the same subject from whom the source material was collected. This is an important safety consideration under CGMP, because an error resulting in the administration of an autologous product to a subject other than that from which the source material was derived could cause serious, life threatening complications.

The central processing method does not provide the manufacturer with control over the (1) personnel, (2) facilities, (3) equipment, (4) documentation, and (5) procedures used at the collection and administration of somatic cell and gene therapy products at the originating facilities where source material is collected or at the facility where a subject is administered the product. When the manufacturer does not control collection of the source material used in an initial phase of the manufacturing process, it is impossible for the manufacturer to guarantee a safe and consistent somatic cell therapy or gene therapy product obtained at an end of the manufacturing process. Consequently, the manufacturer is unable to meet FDA requirements under CGMP regulations when using the central processing system, and is unlikely to meet comparable regulations in other jurisdictions.

The FDA requires that biological materials intended for use as source materials for further manufacture into a licensed somatic cell or gene therapy product have premarketing approval as biological products intended for further manufacture when they are shipped from one legal entity to another (see Federal Register/Vol. 58, No. 1 97/Oct. 14, 1993 notice). Accordingly, manufacturers that use the central processing method will be required to obtain source material only from facilities that are licensed for that purpose. Since the FDA accepts only license applications for production of source material that specify the licensed manufacturer to which the source material will be shipped and only after demonstration of safety and efficacy of the final product, a manufacturer must determine the originating facilities that will be used to manufacture and distribute the product prior to applying for an FDA license. Each entity involved in any portion of the manufacturing process must then submit a license application demonstrating as one of the requirements, CGMP compliance.

Similarly, in the example of an autologous somatic cell or gene therapy product where the manufacturer is required to control the administration site that administers the product to the subject. The manufacturer is required to undertake the costly task of securing an additional government license for each facility conducting the administration portion of the somatic cell therapy or gene therapy production process.

The typical originating and treatment facilities used by manufacturers using the central processing method usually have little to no experience in CGMP drug manufacturing. Therefore, establishing a nationwide or worldwide network of facilities licensed to produce source material for further manufacturing at the central processing facility and administer the final product is a daunting, cumbersome, expensive and impractical undertaking.

CGMP regulations require that manufacturers assure that autologous somatic cell therapy and gene therapy products are administered to the correct subject. Administering the autologous somatic cell therapy or gene therapy product to a subject different from a subject from whom the source material was derived could result in a life-threatening medical condition. There currently are not any CGMP compliant methods for assuring that an autologous somatic cell or gene therapy product is administered to the correct subject.

Another method used to manufacture somatic cell and gene therapy products is the on-site processing method. This method involves the establishment of one or more on-site processing facilities that are typically established in or near a hospital location. This method has the advantage of having all the manufacturing procedures conducted in the same facility. This method is cost-prohibitive when implementing large-scale distribution. For example, either all subjects are required to travel to the on-site processing facility or multiple on-site manufacturing facilities are required to be located near subject population centers. Establishing multiple centers takes away any cost savings associated with scale, as management, QA/QC, personnel and equipment would have to be duplicated at each site. Similar issues arise in other jurisdictions with requirements comparable to those of the FDA.

Therefore, a need exists to develop a system and method for collection, processing and administration of somatic cell therapy products that meet FDA requirements under CGMP regulations. Furthermore, there is a need to develop a cost-effective system and method for manufacturing and distributing somatic cell therapy and gene therapy products under CGMP and/or comparable regulations. In addition, there exists an urgent need to ensure the delivery of autologous somatic cell therapy and gene therapy products that comply with CGMP requirements and comparable requirement to the correct subject.

Thus, among the objects herein, it is an object to solve these and other problems. For example, it is an object to provide methods and systems for production of somatic cell and gene therapy products in which there is vein-to-vein control over collection, processing and administration of somatic cell and gene therapy requirements to meet regulatory guidelines in the United States and elsewhere. It is also object to provide a method of producing and distributing somatic cell and gene therapy products under CGMP under a single manufacturing license. It is also an object herein to provide methods and systems that assure that an autologous somatic cell or gene therapy product is administered to the correct subject. It is another object to provide a cost-effective, regulation-compliant, such as GMP-compliant, method to manufacture and distribute somatic cell therapy and gene therapy products on a large-scale. Other objects and solutions provided herein will be apparent.

### SUMMARY

Provided are methods and systems for producing and distributing somatic cell and gene therapy products so that all steps of the processes are under the control of a manufacturer from vein-to-vein. Such methods and systems should meet current and future regulatory guidelines in the United States and other jurisdictions. The methods and systems for producing and distributing somatic cell and gene therapy products permit manufacture under conditions that meet CGMP under a single manufacturing license. The methods and systems provided herein are designed to ensure that an autologous somatic cell or gene therapy product is administered to the correct subject, to be cost-effective, to be regulatory rule-compliant, such as GMP-compliant, and permit manufacture and distribution of somatic cell therapy and gene therapy products on a large-scale.

The methods provided herein include stablishing a central processing facility and a plurality of satellite facilities all owned and/or controlled by a manufacturer for the collection, processing and administration of a somatic cell therapy or gene therapy product under a single government license. The manufacturer controls a documentation, computer systems and processes conducted at each facility. Systems containing these elements also are provided.

Also provided are methods for ensuring, in the case of an autologous somatic cell therapy or gene therapy, that the final product is administered to the same subject from which the source material was derived, in compliance with governmental regulations, such as CGMP regulations. Systems for effecting these methods also are provided.

In an exemplary embodiment, a method of manufacturing a somatic cell or gene therapy product includes some or all of the steps of: (1) collecting source material at one of the satellite facilities from a donor, (2) transporting the source material from the donor and delivering the source material to a central processing facility, (3) processing the source material from the donor at the central processing facility to produce a somatic cell or gene therapy product under the same single governmental license, (4) transporting the therapy product back to a satellite facility, and (5) administering the therapy product to a subject. The methods and systems include steps and elements for tracking the “chain-of-custody” of a subject source material from collection to infusion in order ensure the product is administered to the same subject from which the source material was obtained. All steps and documentation related thereto are performed under the control of the manufacturer and, for practice in the United States, are designed to be performed under a single government license granted to the manufacturer.

### BRIEF DESCRIPTION OF THE DRAWINGS

FIG. 1 shows a somatic cell or gene therapy system provided herein.

FIG. 2 illustrates a flow chart of an exemplary method of implementing the somatic or gene cell therapy system. Precise steps and protocols can be omitted and added and modified.

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, compliance with good manufacturing practice (CGMP) regulations for devices, biologics and drug, as applicable and as defined under 21 Code of Federal Regulations (CFR) parts 211, 606 and 820 means that a manufacturer to control the (1) facilities, (2) personnel, (3) equipment, (4) documentation, and (5) procedures used during each step involved in manufacturing, distributing and administration of somatic cell and gene therapies. The manufacturer demonstrates compliance with CGMP by providing the FDA with “documented evidence which provides a high degree of assurance that a specific process will consistently produce a product meeting its pre-determined specifications and quality attributes.”

As used herein, vein-to-vein control with reference to a manufacturer's control over somatic cell and gene therapy protocols means that the manufacturer controls all steps and aspects of the procedure(s) from obtaining source material until it is infused into a subject. The manufacturer, thus, controls the (1) facilities, (2) personnel, (3) equipment, (4) documentation, and (5) procedures used during each step involved in manufacturing, distributing and administration of somatic cell and gene therapies from collection of source. As a result of such control a manufacturer can demonstrates compliance with regulatory requirements, such as CGMP as required by FDA in the United States and at least with comparable and less stringent regulations in other jurisdictions.

As used herein, cell therapy is a method of treatment involving the administration of live cells. Adoptive immunotherapy is a treatment process involving removal of cells from a subject, the processing of the cells in some manner ex-vivo and the infusion of the processed cells into the same or different subject as a therapy.

As used herein, the term “somatic cell therapy product” refers to one or more biological cells that have been propagated, expanded, selected, pharmacologically treated or otherwise altered in one or more biological characteristics ex vivo to be administered to an animal, particularly a mammal, such as a human, for prevention, treatment, cure, diagnosis, or mitigation of disease and injury or symptoms of such. A “somatic cell therapy product” can be one or more autologous (self), allogeneic (intra-species), or xenogeneic (inter-species) cell(s) that have been propagated, expanded, selected, pharmacologically treated, or otherwise altered in biological characteristics ex-vivo to be administered to animals, particularly, mammals, such as humans, for the prevention, treatment, cure, diagnosis, or mitigation of disease or injuries or symptoms of such.

As used herein, the term “gene therapy product” refers to one or more products that contain genetic material that are administered to an organism, particularly mammals, such as a human, modify and/or manipulate expression of genetic material and/or alter one or more biological properties of one or more living cells.

As used herein, the terms “autologous somatic cell therapy and gene therapy” refer to somatic and gene products derived from source material collected from a first subject and intended for administration to the same first subject. In addition, the terms “somatic cell therapy product” and “gene therapy product” are referred to as a “therapy product”.

As used herein, source biological material (or source material) is the population of cells that are collected from a subject for further processing into an adoptive immunotherapy. Source material generally is mononuclear cells collected, for example, by leukapheresis. Thus, “source material” refers to a biological material that is capable of being transformed into a somatic cell therapy and/or gene therapy product. Some non-exhaustive examples of source materials include tumor cells, skin cells, tissue, organs, blood or any other biologically-derived material that can be transformed in to a somatic cell therapy and/or gene therapy product.

As used herein, symbology refers to the code, such as a bar code, that is engraved or imprinted on any device or container that contains a subject or other sample. The symbology is any element of a code known or designed by the user. The symbols can be directly read or can be associate, such as by a relational database, with information stored in a remote computer or memory or other such device or means. For example, each sample can be uniquely identified with an encoded symbology. The processing steps can be recorded in a remote computer and associated with the code; the computer can direct the processing and tracking of the sample. The symbology can be read, for example, with an optical reader.

As used herein, a bar codes refers any array optically readable marks of any desired size and shape that are arranged in a reference context or frame of, for example, one or more columns and one or more rows. For purposes herein, the bar code refers to any symbology, not necessarilry “bar” but can include dots, characters or any symbol or symbols.

As used herein, an optical memory refers to the symbology and the surface on which it is engraved or otherwise imprinted.

As used herein, an array refers to a collection of elements, such as different compositoins of cells, containing three or more members. An addressable array is one in which the members of the array are identifiable, typically by position on a solid phase support or by virtue of an identifiable or detectable label, such as by color, fluorescence, electronic signal (i.e. RF, microwave or other frequency that does not substantially alter the interation of the molecules of interest), bar code or other symbology, chemical or other such label.

As used herein, a composition containing “purified cells” means that at least 50%, typically at least 70%, of the cells in the composition are of the identified type. For example, a composition containing purified CD4+ cells is a composition in which at least 50% of the cells in the compositions are CD4+.

As used herein, infusion medium is an isotonic solution suitable for intravenous infusion. Any such medium known to those of skill in the art can be used. Examples of infusion medium include, but are not limited to, normal saline (NS), 5% dextrose (D5W), Ringer's Lactate, Plasma-Lyte and Normosol and any other commercially available medium or medium knonw to one of skill in the art.

As used herein, a professional antigen presenting cells (APC) include dendritic cells, B-cells and macrophages.

As used herein, formulating for infusion is the process of removing or harvesting the cells to be used in adoptive immunotherapy from a culture environment, then subsequently washing, concentrating and re-suspending the cells in infusion medium or in plasma as provided herein.

As used herein, peripheral blood monocytes (PBMC) include autologous and allogeneic cells.

As used herein, culture medium is any medium suitable for supporting the viability, growth, and/or differentiation of mammalian cells ex-vivo. Any such medium known to those of skill in the art. Examples of culture medium include, but are not limited to, X-Vivo15 (BioWhittaker), RPMI 1640, DMEM, Ham's F12, McCoys 5A and Medium 199. The medium can be supplemented with additional ingredients including serum, serum proteins, growth suppressing, and growth promoting substances, such as mitogenic monoclonal antibodies and selective agents for selecting genetically engineered or modified cells.

As used herein, an immunosuppressive tumor environment is the microenvironment created by cytokine production from tumor cells and infiltrating mononuclear cells. The sum total of cytokines create an environment that is capable of suppressing the effector functions of immune cells. Examples of immunosuppressive cytokines in a tumor microenvironment include IL-10 and TGF-beta.

As used herein, a resting T-cell means a T-cell that is not dividing or producing cytokines. Resting T-cells are small (approximately 6-8 microns) in size compared to activated T-cells (approximately 12-15 microns).

As used herein, a primed T-cell is a resting T-cell that has been previously activated at least once and has been removed from the activation stimulus for at least 48 hours. Primed T-cells usually have a memory phenotype.

As used herein, an activated T-cell is a T-cell that has received at least two mitogenic signals. As a result of activation, a T-cell will flux calcium which results in a cascade of events leading to division and cytokine production. Activated T-cells can be identified phenotypically, for example, by virtue of their expression of CD25. Cells that express the IL-2 receptor (CD25) are referred to herein as “activated”. A pure or highly pure population of activated cells typically express greater than 85% positive for CD25.

As used herein, a cell therapeutic refers to the compositions of cells that are formulated as a drug whose active ingredient is wholly or in part a living cell.

As used herein, immune cells are the subset of blood cells known as white blood cells, which include mononuclear cells such as lymphocytes, monocytes, macrophages and granulocytes.

As used herein, T-cells are lymphocytes that express the CD3 antigen.

As used herein, helper cells are CD4+ lymphocytes.

As used herein, regulatory cells are a subset of T-cells, most commonly CD4+ T-cells, that are capable of enhancing or suppressing an immune response. Regulatory immune cells regulate an immune response primarily by virtue of their cytokine secretion profile. Some regulatory immune cells also can act to enhance or suppress an immune response by virtue of antigens expressed on their cell surface and mediate their effects through cell-to-cell contact. Th1 and Th2 cells are examples of regulatory cells.

As used herein, effector cells are immune cells that primarily act to eliminate tumors or pathogens through direct interaction, such as phagocytosis, perforin and/or granulozyme secretion, induction of apoptosis, etc. Effector cells generally require the support of regulatory cells to function and also act as the mediators of delayed type hypersensitivity reactions and cytotoxic functions. Examples of effector cells are B lymphocytes, macrophages, cytotoxic lymphocytes, LAK cells, NK cells and neutrophils.

As used herein, T-cells that produce IFN-gamma, and not IL-4 upon stimulation are referred to as Th1 cells. Cells that produce IL-4, and not IFN-gamma, are referred to as Th2 cells. A method for identifying Th1 cells in a population of cells is to stain the cells internally for IFN-gamma. Th2 cells are commonly identified by internal staining for IL-4. In normal (i.e., subjects not exhibiting overt disease) individuals, generally only about 12 -16% of the CD4+ cells stain positive for internal IFN-gamma after activation; less than 1% stain positive for IFN-gamma prior to activation. It is rare for a T-cell population to stain greater than 35% IFN-gamma positive. The cells resulting from a method described herein (and provided in co-pending U.S. application Ser.529 No. 09/957,194, filed Sep. 19, 2001), stain greater than 70% positive and often greater than 90% positive for IFN-gamma.

As used herein, a pure or highly pure population of Th1 cells is a population that stains greater than 70% positive for internal IFN-gamma and does not produce greater than about 26 pg/ml/10⁶ cells of IL-4 in a 24 hour period. In most instances, they do not produce greater than about 6 pg/ml/10⁶ cells of IL-4 in a 24 hour period.

As used herein, a memory cell is a T-cell that expresses CD45RO and not CD45RA. A pure or highly pure population of memory cells expresses greater than 70%, generally greater than 80%, and even greater than 90% or 95% positive for CD45RO.

As used herein, a cell that has the ability to traffic to a tumor or other site of inflammation upon infusion, is a T-cell with an activated (CD25+) memory (CD45RO+) phenotype that expresses adhesion molecules, such as CD44 and does not expresses CD62L. A pure or highly pure population of memory cells with the ability to traffic to a tumor or other site of inflammation upon infusion is greater than 70%, generally greater than 90% or 95% positive for CD44, and less than about 25%, including less than 5%, positive for CD62L.

As used herein, T-cells intended for adoptive immunotherapy refer to any T-cells that have been treated for use in adoptive immunotherapy. Examples of such cells include any T-cells prepared for adoptive immunotherapy and, include but are not limited to, for example Th1 cells (co-pending U.S. application Ser. No. 09/957,194), co-stimulated T-cells (Lums, et al. (2001) J Immunother 25:408), polyclonal and antigen-specific CTL (Maus et al. (2002) Nat. BiotechnoL 20:143), co-stimulated CD4+ cells (Levine et al. (2002) Nat. Med 8:47), CML-specific T-cells (Muller et al. (2002) J Immunother. 24:482), soluble tumor antigen induced CTL (Li et al. (2001) Zhonghua Wai Ke Za Zhi 39:619), anti-cervical cancer CTL (Chiriva-Internati et al. (2002) Eur. J. Immunol. 32:30), tumor associated lymphocytes (Schuler et al. (2001) J. Exp. Med. 194:1767), EBV-specific T-cells (Savoldo et al. (2002) J. Immunol. 168:909; Hague et al. (2001) Transplantation 72:1399), CML-specific T-cells (Muller et al. (2001) J. Immunother. 24:482), CTL against lung cancer (Hiraki et al. (2001) Anticancer Res.21:2561; So et al. (2001) Jap 579 J Clin. Oncol. 31:31 1), anti-leukemia CTL (Montagna et al. (2001) Blood 98:3359), ex-vivo activated lymph node cells (Plautz et al. (2001) Cancer Chemother Biol Response Modif 19:327), interferon-gamma enhanced T-cells (Becker et al. (2001) Nat Med. 7:1159), pharmacologically-activated lymph node cells (Bear et al. (2001) Cancer Immunol Immunother. 50:269), gamma-delta T-cells (Chen et al. (2001) Int. Arch. Allergy Immunol. 125:256), CMV-specific CTL (Szmania et al. (2001) Blood 98:505; Cho et al. (2001) J. Immunother. 24:242), activated T-cells (Chin et al. (2001) J Surg Res 98:108), pre-immunized effector cells (Morecki et al. (20010 J. Immunother 24:114), cytotoxic T-cells (U.S. Pat. No. 6,255,073; U.S. Pat. No. 5,846,827), expanded tumor draining lymph node cells (U.S. Pat. No. 6,251,385), various preparations of lymphocytes (U.S. Pat. No. 6,194,207; U.S. Pat. No. 5,443,983; U.S. Pat. No. 6,040,180; U.S. Pat. No. 5,766,920; U.S. Pat. No. 6,204,058), CD8+ TIL cells (Figlin et al. (1997) Journal of Urology 158:740), CD4+ T-cells activated with anti-CD3 monoclonal antibody in the presence of IL-2 (Nishimura (1992) J. Immunol. 148:285), T-cells co-activated with anti-CD3 and anti-CD28 in the presence of IL-2 (Garlie et al. (1999) Journal of Immunotherapy 22:336), antigen-specific CD8+ CTL T-cells produced ex-vivo and expanded with anti-CD3 and anti-CD28 monoclonal antibodies (mAb) in the presence of IL-2 (Oelke et al. (2000) Clinical Cancer Research 6:1997), and the first injection of irradiated autologous tumor cells admixed with Bacille Calmette-Guérin (BCG) to vaccinate subjects followed seven days later by recovery of draining lymph node T-cells which are activated with anti-CD3 mAb followed by expansion in IL-2 (Chang et al. (1997) Journal of Clinical Oncology 15:796).

As used herein, activating proteins are molecules that when contacted with a T-cell population cause the cells to proliferate. Reference to activating proteins thus encompasses the combination of proteins that provide the requisite signals, which include an initial priming signal and a second co-stimulatory signal. The first signal requires a single agent, such as anti-CD3 monoclonal antibody (mAb), anti-CD2 mAb, anti-TCR mAb, PHA, PMA, and other such signals. The second signal requires one or more agents, such as anti-CD28 mAb, anti-CD40L, anti-CD99, anti-CD4 mAb, cytokines, feeder cells and other such signals. Thus activating proteins include combinations of molecules including, but are not limited to: cell surface protein specific mAbs, fusion proteins containing ligands for a cell surface protein, or any molecule that specifically interacts with a cell surface receptor on a T-cell and directly or indirectly causes that cell to proliferate.

As used herein, a mitogenic mAb is an activating protein that is a monoclonal antibody specific for a T-cell surface expressed protein that when contacted with a cell directly or indirectly provides one of the at least two requisite signals for T-cell mitogenesis. Suitable mitogenic mAbs induce T-cell doubling times of 24 h to 48 h.

As used herein, a cytokine is a factor produced from a cell that has biological activity. A lymphokine is a cytokine produced by lymphocytes. Interleukins and interferons are examples of lymphokines.

As used herein, exogenous cytokines, refer to cytokines that are added to a sample or cell preparation. They do not include cytokines produced by the cells in a sample or cell preparation in vitro, in vivo or ex vivo. Hence preparing cells in the absence of exogenous cytokines, refers to preparation without adding additional cytokines to those produced by the cells.

As used herein, a composition containing a clinically relevant number or population of immune cells is a composition that contains at least 10⁹, typically greater than 10⁹, at least 10¹⁰ cells, and generally more than 10¹⁰ cells. The number of cells will depend upon the ultimate use for which the composition is intended as will the type of cell. For example, if Th1 cells that are specific for a particular antigen are desired, then the population will contain greater than 70%, generally greater than 80%, 85% and 90-95% of such cells. For uses provided herein, the cells are generally in a volume of a liter or less, can be 500 mis or less, even 250 mls or 100 mls or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells.

As used herein, a clinically relevant number of activated polyclonal Th1 memory cells is a composition containing a clinically relevant number or population of immune cells where a substantial portion, greater than at least about 70%, typically more than 80%, 90%, and 95%, of the immune cells are activated polyclonal Th1 memory cells.

As used herein, polyclonal means cells derived from two or more cells of different ancestry or genetic constitution. A polyclonal T-cell population is a population of T-celis that express a mixture of T cell receptor genes with no one T cell receptor gene dominating the population of cells.

As used herein, predominant means greater than about 50%.

As used herein, highly pure means greater than about 70%, generally greater than 75% and can be as pure as 85%, 90% or 95% or higher in purity. A highly pure population of Th1 cells, as used herein, is typically a population of greater than 95% CD3+, CD4+ T-cells that stain greater than about 70% positive for internal IFN-gamma and do not produce detectable amounts of IL-4 when assayed by ELISA (i.e., less than 26 pg/ml/10⁶ cells). Internal staining for IL-4 is generally below 10% and most often below 5%. Occasionally higher numbers are observed. This is often an artifact of the detection technique, as cells that die by apoptosis will stain positive for internal IL-4. Measurement of secretion into supernatants controls for this artifact. The amount of IFN-gamma detected by ELISA is generally in excess of 1 ng/ml/10⁶ cells and in the range of 1 ng/ml to 26 ng/ml per 10⁶ cells, but can be greater than 26 ng/ml per 10⁶ cells.

As used herein, a combination refers to two component items, such as compositions or mixtures, that are intended for use either together or sequentially. The combination can be provided as a mixture of the components or as separate components packaged or provided together, such as in a kit.

As used herein, colloidal size beads are particles of a size that form a colloid upon mixing with a liquid, such as an aqueous composition. Such particles typically have an a size where the largest dimension is about 0.01 to 2 microns. For purposes herein, it refers to the size of the particles produced in the method of Example 1G.

As used herein, effector cells are mononuclear cells that have the ability to directly eliminate pathogens or tumor cells. Such cells include, but are not limited to, LAK cells, MAK cells and other mononuclear phagocytes, TILs, CTLs and antibody-producing B cells and other such cells.

As used herein, immune balance refers to the normal ratios, and absolute numbers, of various immune cells and their cytokines that are associated with a disease free state. Restoration of immune balance refers to restoration to a condition in which treatment of the disease or disorder is effected whereby the ratios of regulatory immune cell types or their cytokines and numbers or amounts thereof are within normal range or close enough thereto so that symptoms of the treated disease or disorder are ameliorated. The amount of cells to administer can be determined empirically, or, such as by administering aliquots of cells to a subject until the symptoms of the disease or disorder are reduced or eliminated. Generally a first dosage will be at least 10⁹-10¹⁰ cells. In addition, the dosage will vary depending upon treatment sought. As intended herein, about 10⁹ is from about 5×10⁸ up to about 5×10⁹; similarly about 10¹⁰ is from about 5×10⁹ up to about 5×10¹⁰, and so on for each order of magnitude. Dosages refer to the amounts administered in one or in several infusions.

As used herein, therapeutically effective refers to an amount of cells that is sufficient to ameliorate, or in some manner reduce the symptoms associated with a disease. When used with reference to a method, the method is sufficiently effective to ameliorate, or in some manner reduce the symptoms associated with a disease.

As used herein, a subject is an organism, generally an animal, such as a mammal, including a human, in need of treatment for a disease or disorder (i.e. a patient). Treatment of other animals, such as domesticated animals, including cats and dogs, fish and other pets, and farm and zoo animals, such as cows, pigs, sheep, goats, camels, llamas, gorillas, chimpanzees other primates, dolphins and other whales is also contemplated,

As used herein, mononuclear or lymphoid cells (the terms are used interchangeably) include lymphocytes, macrophages, and monocytes that are derived from any tissue or body fluid in which such cells are present. In general lymphoid cells are removed from an individual who is to be treated. The lymphoid cells can be derived from a tumor, peripheral blood, or other tissues, such as the lymph nodes and spleen that contain or produce lymphoid cells.

As used herein, a therapeutically effective number is a clinically relevant number of immune cells that is at least sufficient to achieve a desired therapeutic effect, when such cells are used in a particular method. Typically such number is at least 10⁹, and generally 10¹⁰ or more. The precise number will depend upon the cell type and also the intended target or result and can be determined empirically.

As used herein, a disease characterized by a lack of Th1 cytokine activity refers to a state, disease or condition where the algebraic sum of cytokines in a specific microenvironment in the body or in a lesion(s) or systemically is less than the amount of Th1 cytokines present normally found in such microenvironment or systemically (i.e., in a subjector another such subject prior to onset of such state, disease or condition). The cytokines to assess include IFN-gamma, IL-2, and TNF-alpha. The precise amounts and cytokines to assess depend upon the particular state, disease or condition. Thus, the diseases for which the cells have therapeutic application include, but are not limited to, cancer, infectious diseases, allergic diseases and diseases characterized by overactive humoral immunity (such as in systemic lupus erythematosus).

As used herein, diseases characterized by a Th2-dominated immune response are characterized by either a suppressed cellular immune response or excessive humoral response.

As used herein, a disease characterized by an excess of Th2 cytokine activity refers to a state, disease or condition where the algebraic sum of cytokines in a specific microenvironment in the body or in a lesion(s) or systemically is predominantly of the Th2 type, dominated by IL-4 and/or IL-10 and/or TGF-â. Diseases, states or conditions that exhibit enhanced Th2 responses include infectious diseases such as, but are not limited to, chronic hepatitis C virus infection, leprosy toxoplasmosis infection and AIDS. Imbalance in favor of Th2 cells also occurs in asthma and lupus and other diseases that exhibit suppressed cellular immunity.

As used herein, treatment means any manner in which the symptoms of a condition, disorder or disease are ameliorated or otherwise beneficially altered. Treatment also encompasses any pharmaceutical use of the compositions herein.

As used herein, a vaccine is a composition that provides protection against a viral infection, cancer or other disorder or treatment for a viral infection, cancer or other disorder. Protection against a viral infection, cancer or other disorder will either completely prevent infection or the tumor or other disorder or will reduce the severity or duration of infection, tumor or other disorder if subsequently infected or afflicted with the disorder. Treatment will cause an amelioration in one or more symptoms or a decrease in severity or duration. For purposes herein, a vaccine results from co-infusion (either sequentially or simultaneously) of an antigen and a composition of cells produced by the methods herein.

As used herein, amelioration of the symptoms of a particular disorder by administration of a particular composition refers to any lessening, whether permanent or temporary, lasting or transient that can be attributed to or associated with administration of the composition.

As used herein, substantially pure means sufficiently homogeneous to appear free of readily detectable impurities as determined by standard methods of analysis, such as flow cytometry, used by those of skill in the art to assess such purity, or sufficiently pure such that further purification does not detectably alter the physical and chemical properties, such as biological activities, of the substance. Methods for purification of the immune cells to produce substantially pure populations are known to those of skill in the art. A substantially pure cell population, can, however, be a mixture of subtypes; purity refers to the activity profile of the population. In such instances, further purification might increase the specific activity of the cell population.

As used herein, biological activity refers to the in vivo activities of immune cells or physiological responses that result upon in vivo administration of a cell, composition or other mixture. Biological activity, thus, encompasses therapeutic effects and pharmaceutical activity of such cells, compositions and mixtures.

Although any similar or equivalent methods and materials can be employed in the practice of the methods and cells provided herein, exemplary embodiments are described.

B. Systems and methods

Systems and methods for manufacturing and distributing somatic cell therapy and gene therapy products are provided. For example, systems and methods provided herein permit a user to produce somatic cell therapy and gene therapy products that provide vein-to-vein control by the manufacturer. Such control means that a manufacturer controls all steps and aspects of the procedure(s) from obtaining source material until it is infused into a subject, including tracking of the subject(s) and all documentation. The manufacturer, thus, controls the (1) facilities, (2) personnel, (3) equipment, (4) documentation, and (5) procedures used during each step involved in manufacturing, transporting, distributing and administration of somatic cell and gene therapies from collection of source material through infusion into a recipient subject. In addition, the same manufacturer (1) provides comprehensive documentation and documentation control methods, (2) validates collection, processing, shipping, and product administration procedures, and (3) develops raw material, in-process and final release specifications, methods, procedures and release criteria. For examle, the manufacturer, which controls all facilities and procedures develops documentation that typically includes standard operating procedures (SOP), protocols, master production records, and/or log books that are maintained in the manufacturing facility to provide a chronological record of all equipment-related activities, such as equipment operation, room usage, environmental conditions and cleaning.

In addition, the manufacturer implements a numbering system or other sytstem to identify and track any item in the manufacturing facility that will become a part of, or come in direct contact with, a somatic cell therapy and/or gene therapy product during the manufacturing process when following CGMP regulations. The manufacture implements procedures for tracking donors of source material and recipients of the therapy. The methods provided herein in which a single manufacturer controls one or more central processing facility(ies) for processing the source material and a satellite facility(ies) for obtaining source material and distributing and administering the product, permit such control.

As result of such control as provided by the methods herein, a manufacturer can demonstrate compliance with regulatory requirements, such as CGMP as required by FDA in the United States and at least with comparable and less stringent regulations in other jurisdictions. As a consequence, for example, in the United States manufacture of the product can be performed under a single goverment license. The methods provided herein ensure correct administration of an autologous somatic cell or gene therapy product that meet regulatory guidlines, such as compliance with CGMP regulations in the United States.

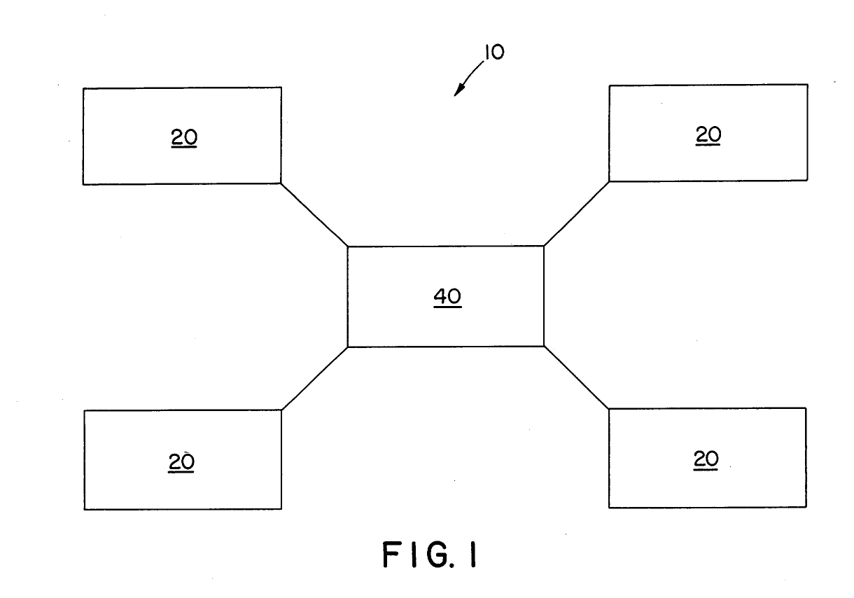

An exemplary system for manufacturing and distributing autologous somatic cell therapy and gene therapy products is generally depicted at 10 in FIG. 1. Though descriptions herein reference an autologous somatic cell therapy and gene therapy product, it is understood that any other somatic cell therapy and gene therapy product, such as allogeneic (intra-species) or xenogeneic (inter-species) somatic cell therapy or gene therapy products, or any combination of any of these, can be substituted in place of exemplified autologous somatic cell therapy and gene therapy products. Likewise, it is to be understood that any combination of any autologous somatic cell therapy and gene therapy product ands any other somatic cell therapy and gene therapy products can be used in the systems and methods provided herein.

The therapy system 10 includes a plurality of satellite centers 20 linked to a central processing facility 40 (or plurality thereof). While the therapy system 10 is illustrated as having four satellite centers 20 linked to the central processing facility 40 (or plurality thereof), it is to be understood that the therapy system can include less than, or more than, four satellite centers 20 to accommodate any steps required by the therapy system 10 when practicing methods provided herein. It is understood that the therapy system 10 can contain a plurality of central processing facilities 40 and each processing facility could be connected to a plurality of satellite centers 20. It is further understood that in the case of a therapy system 10 with a plurality of central processing facilities 40 that individual satellite centers 20 could be connected in a fixed manner to a single central processing center 40 or in a flexible manner to any central processing facility 40 at any given time. As an example, the satellite centers 20 can be connected via a global computer network, such as a secure internet link to the central processing facility 40.

Each satellite center 20 is responsible for collecting source material (not shown) from a subject, such as a cancer patient. Next, the source material is transported from the satellite center 20 to the central processing facility 40 where the source material is processed into a somatic cell therapy or gene therapy product. After processing, the therapy product is transported from the central processing facility 40 back to the satellite center 20 for administration of the therapy product into the subject. An autologous therapy product is administered into a subjectwho provided the source material.

In the therapy system 10, each satellite center 20 is owned and/or controlled by a somatic cell therapy or gene therapy manufacturer (not shown). Each satellite center is controlled by the same manufacturer that controls the central procesing facility. As a result, for example, each satellite center 20 is governed by a single government license secured by the manufacturer to produce the therapy product under appropriate regulations, such as CGMP regulations, or conducts somatic or gene therapy. Furthermore, since each satellite center is owned and/or controlled by same manufacturer, separate government licenses for each satellite center are not required. Therefore, the therapy system 10 permits a user (not shown) to cost-effectively collect source material from a subject and administer the therapeutic product to the subject. In addition, the therapy system 10 permits a user (not shown) to operate the satellite center 20 under the same set of regulations, such as CGMP regulations governed by one government license secured by the manufacturer. Such additional processing steps previously required the collection site to obtain a separate government license to conduct these steps in order to be compliant with CGMP regulations.

Controlling the satellite center 20 under one government license, or other comparable certificate and permits, secured by the manufacturer is advantageous, For example, this permits additional processing steps to be conducted at the satellite center 20 that were not previously possible under CGMP. As an example, the source material (not shown) can be washed, purified, purged, and/or suspended in any number of media in order to condition the source material prior to transport to the central processing center.

Additional processing steps that can be performed include the freezing of the therapy product at the central processing facility 40 prior to shipment to the satellite center 20 for administration to the subject. As a result, in the United States, for example, all steps can be performed under a single government license and in compliance with CGMP regulations. This was not previously possible because freezing at the central processing center required thawing, washing and suspension in infusible or injectable carrier material prior to administration. This processing at the administration site requires a separate government license in the United States and comparable permits in other jurisdictions. The methods and systems herein permit therapy methods to be designed that incorporate these and other processing steps after collection and prior to administration and permit therapy methods to be conducted under the control of a single manufacturer so that, for example, in the United States, only a single goverment license is required. In prior methods, multiple licenses permits are required since no one manufacturer can make the requisite assurances no ensure proper chain-of-custody and tracking of donor and recipient subjects.

The source material or the therapy product can be frozen at the satellite center 20 to extend the shelf life of the source material or the therapy product, if desired, when using the therapy system 10. In prior art central processing methods, the source material or the therapy product could not be frozen nor shipped frozen since freezing is not permissible at a collection site. For exampe, such treatment is not government licensed nor is freezing permissible under CGMP regulations. In addition, freezing the source material or the therapy product in a central processing facility of a prior art central processing method also is not permissible under CGMP regulations. Freezing at the central processing center requires thawing, washing and suspension in infusible or injectable carrier material prior to administration and this processing at the administration site and requires a separate government license. The methods and systems herein permit, therapy methods to be designed that incorporate these and other processing steps after collection and prior to administration that were not previously possible to be conducted in compliance with regulations, such as CGMP regulations under a single government license.

The satellite center 20 can be established near the subject's location in order to conveniently collect source material and/or administer the therapy product to the subject. Furthermore, when the satellite center 20 is used to administer the therapy product to the subject, additional modification of the therapy product can be conducted prior to administration since the satellite center 20 is controlled by the manufacturer and is governed by CGMP regulations. For example, activation of the therapy product like a cell-based therapy product, prior to administration can be desired in order to provide a maximum benefit to the subject. If activation requires the addition of a biological agent to the cell-based therapy product, activation can proceed under FDA-approved procedures that also assure consistent activation and thus, a consistent cell-based therapy product. In another example, sterility testing and/or final product characterization also can be conducted prior to administering the therapy product to the subject. As noted, such procedures can be conducted under CGMP regulations as required by the FDA using the therapy system 10 provided herein.

For example, as described in the EXAMPLES and in copending U.S. application Ser. No. 10/094,667 and attorney docket no. 24731-508B and International PCT application No. (attorney docket no. 24731-508PC, filed the same day herewith) methods are described for solving problems associated with somatic cell and gene therapy products. It is shown in the Examples below and copending applications that cell therapy and gene therapy cell products quickly stop producing high levels of cytokines after harvest. As provided herein and in the co-pending applications, this problem, heretofore not recognized, can be solved by re-activating the cells prior to infusion, and typically after transport to the patient facility and/or at a patient's bedside. The methods herein are well-suited for preparation of and administration of products that are reactivated. Since all facilities and processing steps are under the control of the manufacturer, harvested cells can be frozen or appropriately stored at a central processing facility or a satellite facility and transported to the patient, all under the control of a single manufacturer. Prior to infusion, the cells can be treated to reactivate the cells generally within about 1, 2, 3, or 4 hours of infusion (or other time prior to a significant increase in cytokine production, for example), such as by labeling the cells, for example, with mitogenic mAbs, such as soluble anti-CD3 and anti-CD28 mAbs, and then mixing the labeled cells with autologous mononuclear cells that are optionally enhanced in monocytes and granulocytes. The autologous mononuclear cells act by immobilizing the mitogenic mAbs on the cells, providing an activation stimulus. The mixture of cells is then suspended, for example, in infusion medium (e.g., isotonic solutions such as normal saline, 5% dextrose, Plasma-Lyte (Baxter) and Normasol (Abbott) or, as provided herein, mixed with autologous plasma, and infused into a patient within 24 hours, generally within 4 hours, generally within about 1 hour. If infusion medium is used, it is optionally supplemented with calcium chloride as needed for proper T-cell activation. Alternatively, activation can be effected by mixing the cells with antibody-conjugated colloidal size beads in a suitable infusion medium and treated to reactivate the cells, which are then infused into a subject.

The central processing facility 40 processes the source material into a therapy product. The central processing facility 40 further can include, a management information system (not shown) that permits each satellite center to connect and/or communicate with the central processing facility 40. The central processing facility 40 also is controlled and/or owned by the manufacturer in the therapy system 10. In addition, since the same manufacturer controls the central processing facility and satellite facilities, in the United States, processing of the source material at the central processing facility 40 is conducted under CGMP regulations. Furthermore, an entire manufacturing process, from collection of the source material to administration of the therapy product, can be accomplished under the single government license. In addition, the therapy system 1 0 provides a manufacturing process that is conducted under CGMP regulations that meet FDA or other world-wide regulatory agency requirements. The therapy system 10 also permits cost-effective control of the entire manufacturing process by the manufacturer.

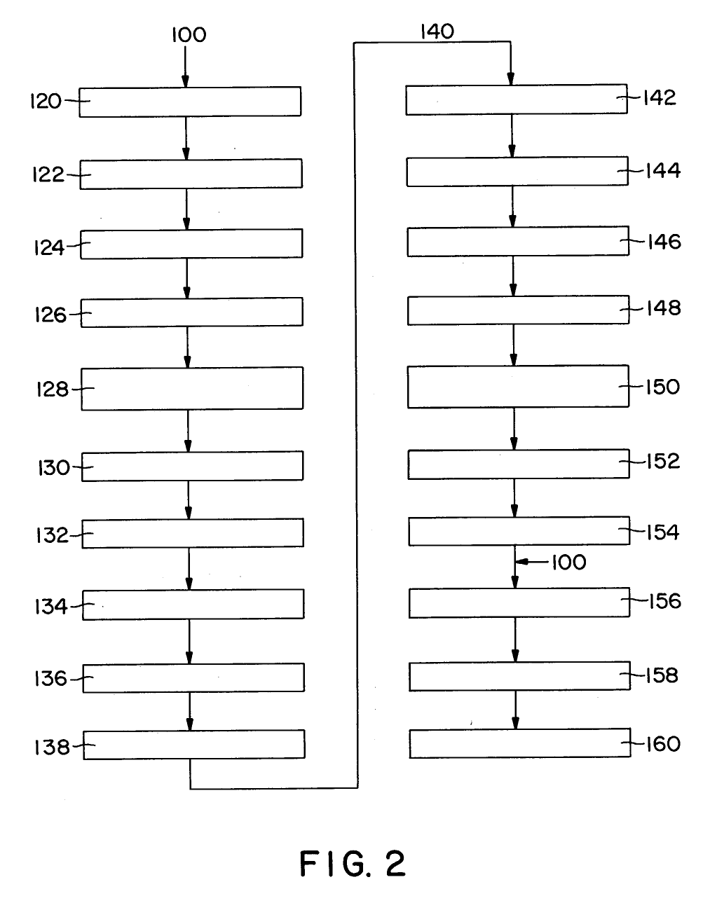

An exemplary method of implementing the therapy system 10 provided herein is generally depicted at 100 in FIG. 2. The indicated steps can be performed in another order, and some steps optionally not performed, depending upon the regulatory requirements of a jurisdication. The following discussion references FDA requirements for CGMP practice, but it is understood that modifications to method can be adapted to conform to different regulatory requirement outside the United States and in the United States if the requirements change.

An exemplary protocol and procedures are depicted in FIG. 2. It is understood that these precise protocols are exemplary of tracking a procedures can be modified as needed. According to an exemplified method, a subject 100 logs in at the satellite facility (step 120) that is administered under a single government license to conduct somatic cell and gene therapy. Next, an administrator (not shown) collects subject specific identification information, such as name, address, phone numbers, emergency contact information, social security number, driver's license number, for example, and enters this information into a database (step 122). After entering the information into the database, the administrator photographs a subject(step 124) to generate a digital photograph of the subject. Next, a subject's information (step 122) and the digital photograph (step 124) of the subject 100 are transmitted to a central database (step 126) located at the central processing facility 140. The a subject's information and digital photograph, for example, can be transmitted (step 126) through a secure internet connection to the central processing facility 140 when practicing the methods provided herein.

Though the descriptions herein reference use of a secure internet connection to transmit a subject's information and the digital photograph to the central database (step 126), it is understood that any other method of storing and/or transporting a subject's information including the digital photograph, such as computer disk, CD-ROM, fax, courier or land mail, or any combination of any of these, can be substituted in place of the secure internet connection while still realizing benefits of the methods and systems provided herein Likewise, it is to be understood that any combination of the use of a secure internet connection and any other method of storing and/or transporting a subject's information can be used.

After transmitting a subject's information and photograph to the central database (step 126), a global unique identifier, such as an identification barcode (GUID), can be assigned to a subject's information and digital photograph (step 128). Next, the identified, such as a GUID barcode, is printed onto an identification badge along with a subject's name and photo. In addition, a label or other identifier, such as a barcode label, is printed (step 132) and used to label a source bag designed to contain a source material that is collected from the subject (step 134).

Next, source material collected from a subject is placed in the labeled source bag (step 136), also referred to as a “parent” source bag and transported to the central processing facility 140. Upon arrival at the central processing facility 140, a central processing facility administrator logs in the “parent” source bag (step 142). The central processing facility 140 also is under the control of the same manufactures. As a result, it can be controlled by the same permits, so that, for example in the United States, it is administered under the same single government license as the satellite facility. The parent source bag is scanned and an identifiable label, such as a GUID barcode label that is identical to the GUID barcode label on the “parent” source bag, is generated as part of the log-in procedure. Next, the label, such as a barcode label, is printed and used to label a “child” source bag (step 144), thereby tracking the material.

After labeling the “child” source bag, the subject source material is transferred into the “child” source bag (step 146). This procedure is repeated each time the process requires transfer of a subject's cells from one holding container to another. The subject source material in the “child” source bag can be processed into the therapy product (step 148) or processed into the therapy product after transfer for culturing or other treatment, such as expansion in a bioreactor (not shown). After producing the therapy product (step 148), a label, such as GUID barcode label, is printed and used to label a therapy product source bag (step 150). Next, the therapy product can be transferred from the “child”s source bag or the bio-reactor into the labeled therapy product source bag (step 152).

After transferring the therapy product into the therapy product source bag (step 152), the therapy product source bag is transported to the satellite facility (step 154). The satellite facility can be the same, or a different satellite facility from the on that the subject 100 used to submit the source material (step 120) as long as the satellite facility is controlled by the same manufacturer with the same protocols and procedures as the cetnral processing facility 140, so that, for example, it can be administered under the single government license that also is used to administer the central processing facility 140.

Next, the therapy product source bag is logged into a satellite facility (step 156). In addition, a subject 100 arrives at the satellite facility and logs in with an administrator (step 156). The therapy product source bag and the identity of the subject are verified using the labels, such as, GUID barcode labels, on the source bag and the badge of the subject (step 158). The labels, such as GUID barcode labels, on the therapy product source bag and the badge are entered, such as by scanning, into a computer work station connected to a central database and the database displays the photograph associated with the scanned labels (step 158), such as GUID barcode labels. A match of the photograph on the badge of the subject and the photograph displayed from the database is verified to match the subject by at least two satellite center employees, such as, for example, a nurse and a QC representative. The verification can be documented by electronic signature (step 158). For privacy reasons, if needed, the digital photograph of the subject 200 can be designed so that it is only viewable from the database by the nurse or other individual actually in the presence of the subject.

After verifying that the subject 100 is the source of the source material used to produce the therapy product, and the therapy product source bag contains the therapy product derived from the source material of the subject 100, the therapy product is administered to the subject 100 (step 160). As an example, the therapy product can be infused to the subject (step 160).

Using a method provided herein, each step, from collection of the source material to administration of the infusion product is conducted under to the control of the same manufacture in accord with the same procedures, so that, for example, the process is in strict compliance with CGMP. In addition, the method permits thawing and formulation of the therapy product at a subject's location in accord with relevant regulations and procedures, such as under CGMP. The methods herein permit scale-up of the manufacturing process of the therapy product by adding a plurality of satellite centers 220 in subject population areas around the world. The connection of each satellite center with the central processing facility, or a different satellite center, provides cost-effective production of therapy products that comply with a jurisdiction's regulations, such as under CGMP regulations in the United States.

In addition, the systems and methods permit tracking of multiple subject source materials, such as by imprinting symbologies on subject samples, through a complex process without risking the administration of the therapy product to a wrong subject. Furthermore, collection, processing and administration can be conducted on the same day or a future day so long as the source material and corresponding therapy product remain viable for treatment of disease.

C. Collection of cells at Satellite facilities and Processing of cells at the central processing facility

The source biological material is processed at the cental processing facility to produce a thereapy product, such as a somatic cell or gene therapy product. These therapy products include, but are not limited to, compositoins of T-cells, such as compositions of substantially purified (i e., at least about 70%, typically at least about 90% of the cells are of the specified type) Th1 cells, substantially purified Th2 cells, and subtypes (i.e., memory cells) thereof; compositions of TIL (tumor infiltrating lymphocytes) cells and other immune cells treated to render them effective as gene therapy products are contemplated (see, e.g., pending U.S. application Ser. Nos. 08/700,565, 09/127,411, 09/127,142, 09/127,138, 09/127,141, 09/824,906, and International PCT application No. WO 97/05239; U.S. application Ser. No. 10/071,016; U.S. application Ser. No. 09/957,194; and U.S. application Ser. No. 10/094,667; U.S. Pat. Nos. 5,872,222, 6,352,694 and others). The cells can be genetically modified for delivery of therapeutic gene products by gene therapy or to enhance or alter the functions or properties of the cells. Any composition of cells, including T cell compositions, for any therapeutic protocol, can be processed by the methods herein, including re-activation before infusion as described herein and in copending U.S. application Ser. No. 10/094,667 and attorney docket no. 24731-508B and International PCT application No. (attorney docket no. 24731-508PC, filed the same day herewith).

1. Collection of source material and Preparation of Th1 cells

As discussed above, a subject is processed at a satellite facility and source material, such as blood or plasma or other such body fluid, is collected for processing either for re-infusion or for treatment of another recipient. Processing is performed at the central processing facility, but can be performed at one or more satellite facilities instead of or in addition to the central processing facility.

For exemplary purposes, disclosure from U.S. application Ser. No. 10/071,016 and U.S. application Ser. No. 09/957,194, which describe preparation of compositions of highly pure population of polyclonal Th1 memory cells, for cell therapy is reproduced herein. This understood to be exemplary of preparation compositions of cells that can be processed in a accord with the methods and systems provided herein in which the manufacturer controls the process vein-to-vein (i.e. from the donor to the recipient).

The methods exemplified and described are for consistently producing a population of highly pure, activated, polyclonal memory Th1 cells from a subject blood sample in the absence of any exogenous growth or differentiation factors (such as IL-2 or IFN-y) for use in adoptive immunotherapy. The methods include the steps of: (i) the collection of source material from a subject, such as at a satellite facility in accord with the procesing steps described above, and transport to the central processing facility as described above; (ii) the purification of T-cells from the source material to produce a therapy product at the central processing facility, and the frequent (every 2-3 days) activation of the purified T-cells and typically repeated (a minimum of 3 times) and return of the product to a satellite facility; (iii); and optionally (iv) the reinfusion of the resulting cells into the same subject at the satellite or other facillity under control of the manufacturer.

a. Source Cell Collection

In practicing a method provided herein, at the satellite facility, a starting population of mononuclear cells is collected from a subject, such as by leukapheresis, in order to obtain the greatest starting cell population number. This is the source material, which is then transported to the central processing facility for production of a gene therapy product therefrom. The cells are transported under the control of the manufacturer and in accord with the manufacturer procedures and documented and tracked in accord with such procedures to an other facility for processing.

For example, a population of CD3+ T-cells, generally CD4+ cells, is then purified from the source population of mononuclear cells. Purities should be in excess of 90%. These are the starting population of cells. The CD4+ cells can be purified by positive selection as more fully explained below. In subjects with large numbers of Th2 cells resident in the memory cell population (CD45RO+),the CD4+ cells can be further purified in order to obtain a starting population of only naive CD4+ cells. This is accomplished by purging the CD4+ cells of CD45RO+ cells. Purified CD4+ cells express CD45RA+ and CD62L^{hi} surface antigens and produce IL-2 upon activation. CD4+ cell populations purified and activated as provided herein contain few, if any, IL-4 producers and also fail to initially make substantial amounts of IFN-y. The methods provided herein are capable of producing a pure population of activated Th1 memory cells from a starting population of CD4+ cells, as well as capable of enhancing the population of activated Th1 memory cells from starting populations of CD3+ cells and CD4+,CD45RO+ cells. It is known that CD4+ cells can develop into cells that principally produce IL-4 or IFN-y upon restimulation. Prior methods use exogenous cytokines to cause this differentiation ex vivo.

b. Initial Activation

The starting cells must undergo an activation step in order to develop into Th1 cells. Generally it is known that CD4+ cells can be activated by antigen presented on MHC Class II molecules or polyclonal stimulants such as Con A, PMA or anti-CD3. For purposes of herein, an exemplary method of activation method is immobilized anti-CD3/anti-CD28 mAb costimulation. In order to assure the differentiation of Th1 cells after activation, the concentration of IL-4 at the time of activation has to be extremely low or even non-existent. IL-4 is known to have a profound effect on the ability of the CD4+ cells to differentiate into Th2 cells. For example, activation of CD4+ cells in the presence of IL-4 concentrations of as little as 50 pg/ml is enough to cause the population of Th2 cells in the culture to increase greater than 100-fold. This increase is known to be due to differentiation of CD4+ cells into Th2 cells and not the expansion of pre-existing Th2 cells. Therefore, it is important to assure that the starting population of cells collected for the purpose of ex-vivo differentiation of Th1 cells are purged of all cells that are producing IL-4. Failure to purge IL-4 producing cells prior to the initial activation will result in Th2 cell contamination of the final product.

c. Initial Purification

Because the starting population of cells must be activated in the absence of IL-4 in order to prevent Th2 differentiation, the cellular sources of IL-4 must be first purged from the starting culture. The cellular source of the early burst of IL-4 that drives Th2 differentiation in-vivo has not been conclusively identified. Therefore, the exact cell types necessary to purge from the starting culture is not clear. Among the cell types that for which purging is recommended are CD117 + granulocytes, basophils, NK cells, and NK1.1 T-cells, which are sources of IL-4 (see, Wang et al. (1999) Clinical Immunology 90:47; Poorafshar et al. (2000) European Journal of Immunology 30:2660; Singh et al. (1999) Journal of Immunology 163:2373; Leite-De-Moraes et al. (1998) European Journal of Immunology 28:1507; Poynteret al. (1997) Cellular Immunology 179:22). So at least these subsets of cell are purged from the starting culture.

Immune cell subsets can be purged using monoclonal antibodies specific for unique cell surface molecules on the target cells. To isolate cells, they can be indirectly stained with specific biotinylated antibody and passed through a avidin-coated column (Handgretinger et al. (1994) Journal of Clinical Laboratory Analysis 8:443) or the antibodies can be immobilized on immunomagnetic beads or particles directly, mixed with the cells and placed under a magnetic field (Mantovani et al. (1 989) Bollettino—Societa Italiana Biologia Sperimentale 65:967; Jacobs et al. (1993) Research in Immunology 144:141; Partington et al. (1999) Journal of Immunological Methods 223:195). Alternatively, the cells can be labeled with the monoclonal antibody and mixed with immunomagnetic particles coated with species-specific antibodies that bind to the monoclonal antibody specific for the cell surface marker (indirect method) (Hansel et al. (1989) Journal of Immunological Methods 122:97). Immobilizing the monoclonal antibody to a solid surface, such as a culture flask (panning) can also be used (Prince et al. (1993) Journal of Immunological Methods 165:139), as well as florescent-activated cell sorting techniques.

Negative selection can be performed with a cocktail of monoclonal antibodies (mAb) specific for cell surface markers that are exclusively expressed on the unwanted cells. For example, for purging the cells herein, a cocktail containing mAbs to CD19 (B-cells), CD56 (NK cells), CD14 (monocytes/macrophages) and CD8 (cytotoxic T-cells) was used to obtain a population of pure CD4 cells by negative selection. This cocktail when used with immunomagnetic beads results in a pure population of CD4+ cells (>95%) when the cells are derived from normal donors.

For purposes herein, however, negative selection purification techniques are not desirable for purification of the source cells. Negative selection leads to an unknown starting population of cells that can negatively affect the purity of the final product. Subjects with immunologically-mediated diseases, and cancer subjects in particular, present with a wide variety of hematological profiles. Subject blood can have many immature cells with altered surface expression so it is difficult to define a monoclonal antibody cocktail that can purge all unwanted cells from a mononuclear cell sample from every subject. These unidentified cells can contaminate the starting cell population. The same mAb cocktail that results in a pure population of CD4 cells from normal donors, when used on blood samples from cancer subjects, results in CD4 cells with very poor purity (only 30-60% CD4+). The poor purity of the starting population of cells prevents the generation of a high purity final product of Th1 cells.

Therefore, in embodiments herein a positive selection protocol is used in order to isolate pure populations of CD4 cells from subject blood. Positive selection allows the retention of only the desired CD4+ cells, while all the unwanted contaminating cells, of known and unknown phenotypes, are purged from the culture. A method for positive selection is to use an anti-CD4 mAb conjugated to immunomagnetic beads or magnetic particles in order to positively select CD4+ cells from the source subject blood samples.

Purification of source cells is rarely used in prior adoptive immunotherapy methods and when it is used, negative selection protocols have been preferred. Positive selection is not often used to purify immune cell subsets due to the difficulty of removing the selected cells from the beads after the selection. Physically removing the cells from the beads by gentle agitation results in very pure CD4 cells (greater than 95% CD4+), it also results in a lower yield than negative selection techniques (yields of 50-60% compared to greater than 70% using positive selection). Another problem with positive selection is that significant numbers of cells retain mAb on their CD4 receptors or internalize their CD4 receptors after selection, making it difficult to access the purity of the cells by FACS. This can be solved by waiting 24-48 h before analysis or by staining for CD3+, CD8− cells as an indirect determination of CD4+ cells.

Another reason why positive selection has not been used to purify T-cells, especially CD4+ T-cells, from source material is that such techniques have technical problems when being applied to source material derived from cancer subjects. The positive selection of CD4+ cells directly from mononuclear cells isolated from cancer subjects often lead to a massive loss of viability of the selected CD4 cells. This does not occur when the same positive selection techniques are applied to source material from normal donors. Some macrophages are known to express the CD4 surface marker, it appears that the purification process activates these macrophages causing them to produce a substance that is lethal to CD4+ T-cells. Since cancer subjects have been exposed to many different chemotherapy drugs and radiation treatments, this could predispose the macrophages to produce a lethal substance upon ligation of the CD4 molecule. Accordingly, when practicing the methods herein with cancer subjects, the macrophage component of the source cell population should be minimized prior to the CD4 positive selection step.

An exemplary method to reduce the macrophage population is to first incubate the collected mononuclear cells overnight on plastic. This takes advantage of the well known property of macrophages to adhere to a surface. The next morning, the non-adherent fraction of cells can be collected and subjected to positive selection of CD4 cells. Another method is to pass the mononuclear cells through a column of nylon wool prior to CD4 positive selection. Macrophages attach to the nylon wool fibers and are thus removed from the culture. The use of macrophage-specific mAbs and complement can also be used.

Prior removal of the adherent fraction of mononuclear cells enabled CD4 cells to be positively selected from cancer subject mononuclear blood samples without loss of viability.

d. Differentiation of Th1 cells

Activation in the presence of IFN-y and the absence of IL-4 is required to cause CD4+ to differentiate into Th1 cells. Advantageously, methods exemplified herein do not require the addition of any cytokines. Also, the methods do not require the presence of macrophages for differentiation, which play a critical role in directing CD4+ cells to differentiate into Th1 or Th2 cells. Macrophages, however, are short-lived in cultures, and thus limit the applicability of methods and compositions that rely macrophages for differentiation. The methods herein, thus, avoid this.

The initial activation of purified CD4+ cells with immobilized anti-CD3 and anti-CD28 induces the cells to produce IL-2 and no IFN-Y. Without further stimulation, the cells expand and differentiate into mixed populations of Th1 and Th2 cells. When the CD4 cells are derived from cancer subject blood, there is sometimes production of detectable amounts of IL-4 in the cultures after the initial activation with anti-CD3/anti-CD28. CD4 cells positively selected after depletion of non-adherent monocytes are known to produce IL-4 (Stanciu et al. (1996) J. Immunolog. Methods 187:107-115).

When IL-4 is detected after the initial activation, a significant amount of the IL-4 was found to be produced by the memory CD4+, CD45RO + subpopulation of the starting cells. Others have also identified memory cells as a source of IL-4 (Sasama et al. (1998) International Archives of Allergy and Immunology 117:255).

Because of the Th1/Th2 imbalance in cancer subjects and in other subjects with diseases in which the Th2 phenotype predominates, the memory cell subset of CD4+ cells is enriched in IL-4 producing cells. Therefore, it may be necessary to also purge the CD45RO+ cells from the starting cells to enhance the purity of the final population of Th1 cells. The necessity for this purging step can be determined empirically for a particular subject or disease state, or the step can be routinely included to ensure that such cells, if present, are eliminated.

As described herein, the method provided herein that employs frequent activation with immobilized anti-CD3/anti-CD28 can cause such high amounts of endogenous IFN-y production from the culture that any contaminating cells with the capacity to produce IL-4 are inhibited. Therefore, while small amounts of IL-4 may be detectable in the early activation steps, IL-4 production becomes negligible after several rounds of activation with anti-CD3/anti-CD28. Therefore, it is rarely required that the CD45RO+ population needs to be purged from the starting cells, even when the source cells are derived from cancer subjects.

If the CD45RO purge step is performed, additional technical issues need to be addressed. After collection of mononuclear cells by leukapheresis, if the CD4 positive selection is performed prior to the CD45RO purge, there is a significant loss of yield. This is because residual mAb on CD4 cells causes CD4 cells to be purged with the CD45RO cells. For this reason, in one embodiment the macrophage fraction removed first, and the CD45RO+ cells are purged by negative selection followed by positive selection for CD4+ cells. This results in a pure population of viable CD4+, CD45RA+ naive T-cells (pTh cells).

When processing cancer subject blood, the CD45RO purge step followed by the CD4 positive selection often results in viable cells, even without the macrophage reduction step. This is due to the significant loss of adherent cells during the CD45RO negative selection process. For the most consistent production of Th1 cells from a variety of subject blood, the purge the macrophage population prior to purification of the CD4 or pTh cells should be performed.

Unlike prior methods, the purified pTh or CD4 cells can be caused to differentiate into pure populations of Th1 cells without addition of exogenous cytokines. Activation of pTh cells by a variety of methods, including anti-CD3/anti-CD28, is known to result in the differentiation of Th2 cells. Naive CD4+ cells are a significant source of IL-4 (Noben-Trauth et al. (2000) Journal of Immunology 165:3620; Demeure et al. (1995) European Journal of Immunology 25:2722). It has been reported that almost every single naive human CD4 T cell primed and expanded in the absence of exogenous IL-4 releases sufficient autocrine IL-4 to support differentiation into Th2 cells (Yang et al. (1995) European Journal of Immunology 25:3517).

It was found herein, however, that when pTh cells or CD4+ cells were repeatedly and frequently (about every 2-3 days) activated with anti-CD3/anti-CD28 that they do not produce IL-4. Upon each stimulation, the cells produced increasing amounts of IFN-y. In particular, it is shown herein, that when pTh cells or CD4 cells are repeatedly (minimum of 3 times) and frequently (every 2-3 days) activated with anti-CD3/anti-CD28 that they do not produce IL-4. Upon each stimulation, the cells produce increasing amounts of IFN-y. The repeated activation causes such large amounts of IFN-y to be produced that it compensates for a poor quality initial purification and still resulting in highly pure Th1 memory cells at the end of the process. The large amounts of IFN-y produced into the culture act to inhibit any production of IL-4 by contaminating cells. Reactivation at a frequency of every 2-3 days for a period of about 9-14 days consistently results in the differentiation of highly pure populations of Th1 memory cells even if the starting population is CD3+ T-cells (CD4+ cells contaminated with CD8+ cells; see, EXAMPLES).

e. Expansion Without IL-2

CD4 cells purified from cancer subjects and activated with immobilized anti-CD3/anti-CD28 do not expand efficiently without the addition of exogenous IL-2. It is known that T-cells from normal donors expand without exogenous IL-2 after being stimulated with anti-CD3/anti-CD28 (see, (Ledbetter et al. (1985) Journal of Immunology 135:2331; Levine et al. (1997) Transplantation Proceedings 29:2028). When the cells are derived from cancer subject blood, however, the addition of exogenous IL-2 is required to create optimal growth conditions for anti-CD3/anti-CD28 activated T-cells from cancer subjects (Garlie et al. (1 999) Journal of Immunotherapy 22:336). There are no reports of successful expansion of cancer-derived T-cells without the use of exogenous IL-2.

Source cells from cancer subjects were found to contain significant amounts of TGF-beta. TGF-beta is known to down regulate T-cell proliferation. Significant amounts of the TGF-beta appear to originate from platelets, which are a known source of TGF-beta (Werz et al. (1996) Pharmazie 51:893). Processing of subject blood causes the release of significant amounts of TGF-beta presumably from the platelets, whereas TGF-beta release is not evident in cultures of processed normal blood. It is not known why the platelets from cancer subjects release TGF-beta during processing, but it may be related to the effect of radiation and chemotherapeutic drugs on the fragility of the platelets. Increased plasma levels of TGF-beta have been reported in subjects with cancer (Jiang et al. (1995) Acta Haematologica 94:1).

Accordingly, the platelet population is reduced in the collected mononuclear cells prior to any processing. This can be achieved, for example, by centrifuging the collected mononuclear cells, such as centrifugation for about 2-5 minutes at 150×g, followed by purging the platelet rich supernatant. Purging platelets from the starting population of mononuclear cells permits cancer subject T-cells to be efficiently expanded with anti-CD3/anti-CD28 mAb without the requirement for exogenous IL-2 addition.

The isolation of pure CD4+ T-cells from subject blood, and the subsequent activation of the cells repeatedly with immobilized anti-CD3 and anti-CD28 mAb results in the expansion of these cells without exogenous cytokines and consistently generates activated Th1 memory cells with high purity. These resulting Th1 memory cells produce large amounts of IFN-y and no detectable IL-4 and express an activated memory phenotype (CD3+, CD4+, CD45RO+, CD62L-, CD25+, CD44+).

The cells are then packaged for shipment, such as by freezing or formulation in suitable medium or in the bags or bioreactors in which they are grown, and shipped to a satellite facility.

2. Reactivating the cells at the satellite facility prior to infusion and formulation thereof for infusion

In this exemplary protocol, the cells are processed at a different facility from the satellite facility at which they are collected. Then are then transported under the control of the manufacturer and in accord with the manufacturer procedures and documented and tracked in accord with such procedures. The cells are then optionally tested or further processed.

For example, it has been found (see, e.g., copending U.S. application Ser. No. 10/094,667 and attorney docket no. 24731-508B, filed the same day herewith see, also International PCT application No. attorney docket no. 24731-508PC, filed the same day herewith) that it may be necessary, and is generally advantageous, to re-activate cells intended for cell therapy prior to infusion. Prior to such reinfusion, after processing, the therapy product cells transported back to the satellite facility. The cells, hence are rested; the total period for rest can be for the transport period and is generally between 24 and 120 hours, typically it is between about 72 hours and 96 hours. Following this rest period the cells are re-activated prior to infusion, generally 1 hour to 1 day, typically 1 to 8 hours prior to infusion, of the cells into a patient.

It is contemplated herein that any method for activation of T-cells may be used just prior to infusion. Such activation should be performed no more than about 24 hrs, and is typically 8, 6, or 4 hours before infusion. The best time for infusion, can be determined empirically and should be after the cells are activated but before cytokine production increases substantially, since infusion of cells that are producing large amounts of cytokines may be toxic. This timing can be determined empirically by activating the cells and measuring cytokine production as a function of time. For the exemplified cells this time period is about 4 hours after activation (see, e.g., EXAMPLES, for an exemplary time course).

a. General methods for activating T-cells

In order for T-cells to proliferate, they require two separate signals. The first signal is generally delivered through the CD3/TCR antigen complex on the surface of the cells, and the second is generally provided through the IL-2 receptor. For cells used in adoptive immunotherapy, IL-2 is generally used as the second signal. In order to bypass the IL-2 signal, combinations of mAb can be used for activation. The mAb can be in, the soluble phase or immobilized on plastic or other solid surfaces such as on magnetic beads.

i. First signal

To provide the first signal, cells are generally activated with mAb to the CD3/TCR complex, but other suitable signals, such as, but not limited to, antigens, super antigens, polyclonal activators, anti-CD2 and anti-TCR antibodies, can be used. Other suitable agents can be empirically identified. Immobilized or cross-linked anti-CD3 mAb, such as OKT3 or 64.1, can activate T-cells in a polyclonal manner (see, Tax, et al. (1983) Nature 304:445). Other polyclonal activators, however, such as phorbol myristate acetate can also be used (see, e.g., Hansen, et al. (1980) Immunogenetics 10:247).

Monovalent anti-CD3 mAb in the soluble phase can also be used to activate T-cells (see, Tamura et al. (1992) J. ImmunoL 148:2370). Stimulation of CD4+ cells with monovalent anti-CD3 mAb in the soluble form is typically used for expansion of Th2 cells, but not Th1 cells (see, dejong, et al. (1992) J. Immunol. 149:2795). Soluble heteroconjugates of anti-CD3 and anti-T-cell surface antigen mAb can preferentially activate a particular T-cell subset (see, e.g., Ledbetter, et al. (1988) Eur. S. Immunol. 18:525). Anti-CD2 mAb can also activate T-cells (see, Huet, et al. (1986) J. Immunol. 137:1420). Anti-MHC class II mAb can have a synergistic effect with anti-CD3 in inducing T-cell proliferation (see, Spertini et al. (1992) J. ImmunoL 149:65). Anti-CD44 mAb can activate T-cells in a fashion similar to anti-CD3 mAb. See, Galandrini, et al. (1993) J. Immunol. 150:4225)

ii. Second signal

A variety of mAb singly or in combination can provide the second signal for T-cell activation. Immobilized mAb or fusion proteins which interact with co-stimulatory molecules such as CD28, CD134 (OX40) and CD137 (4-1BB) or adhesion molecules on T-cells such as CD54 (ICAM-1), CD11a/CD 18 (LFA- 1) and CD49d/CD29 (VLA-4) singly or in combination can provide second signals for activation.

To determine the combination of mAbs or proteins that optimally induce sustained regulatory cell proliferation, a screening procedure using combinations of these mAbs or proteins is used. The cells are incubated with various combinations of these substances and screened for growth by analysis of ³H-thymidine incorporation or equivalent methods. The group demonstrating the best growth characteristics is selected for use.

b. Exemplary methods for re-activating the T-cells prior to infusion

Any method for activating T-cells can be employed to re-activate the cells. In most instances, since the cells are to be reactivated at the patient bedside or on site, the method must be conducted in a manner the maintains sterile conditions, such as those required by Good Manufacturing Practices (GMP). In accord with the methods herein, the re-activation is performed under the conditions and control as all other steps. Methods for reactivation are exemplified herein. Typically these methods are performed at the satellite facility under control of the manufacturer.

i) In one method, a patient is leukapheresed, and mononuclear cells, which are enriched in granulocytes and monocytes, are collected. At same time, the frozen cells are labeled with anti-CD3/CD28 antibodies, preferably IgG1, mixed with the enriched mononuclear cells. The granulocytes and monocytes have Fc receptors that bind with high avidity to Fc portion of IGg1. Therefore they deliver a signal to the cells, activating them. The resulting cytokine profile from the cells is another log higher than when they are activated with bead-bound monoclonal antibodies. In addition, the cells activate the monocytes and granulocytes to produce cytokines, such as IL-12, which are macrophage, not T-cell, products.

The resulting mixture of cells produce so much cytokine that they could be cytotoxic. It was found, however that there are no measurable cytokines within the first 4 hours of activation, and that the peak of cytokine production is at 24 hrs. Therefore, the cell composition is infused within four hours after activation. If, for example, the cells are memory cells (see, e.g., co-pending U.S. application Ser. No. 09/957,194), they traffic to tumors and sites of inflammation, and start producing cytokines at the targeted site(s).

ii) Another method for activating T-cells for use in adoptive immunotherapy protocols is to incubate the cells with immunomagnetic beads conjugated with anti-CD3/anti-CD28 mAbs. Cells activated in this manner must be removed from the beads prior to infusion, as the beads are not intended for human infusion. Typically, the conjugated beads are separated from the cells using a magnet. The initial interaction between the conjugated beads and the cells in strong. Attempts to remove the conjugated beads from the cells within 24 hours, results in significant cell death, presumably due to damage to the cell membranes as the beads are pulled off the cells. After 24 hours, and preferably after 48 hours, the interaction between the conjugated beads and the cells weaken and the cells can be readily separated without significant loss of viability. However, cells that are removed from the conjugated beads after 24-48 hours produce diminished amounts of cytokines.

iii) In accord with the methods provided herein, activted T-cells are removed from the conjugated beads after 48 hours and incubated without activating stimulus for an additional 24-48 hours. When these resting cells are reactivated, they produce at least about 2-10-fold, generally at least about 5-20-fold, more cytokine than cells that were not rested and reactivated. In addition, rested and reactivated cells continue to produce cytokines for at least 96 hours after restimulation. Non-rested, stimulated cells only produce cytokines for 48 hours.

Thus, as provided herein, to advantageously employ cells for adoptive immunotherapy protocols, cells are reactivated just prior to infusion into a patient. Reactivation can be effected by any method of activation. Mitogenic mAbs, however, require immobilization in order to deliver an activation signal to T-cells, which is provided by beads with immobilized antibodies. Conjugated beads cannot be used for activation prior to reinfusion, since they readily can not be removed when added just prior to infusion and conjugated beads can not be infused in high quantity to a patient.

Accordingly, an alternative activation method that does not require removal of beads can be used. Immobilization of mitogenic mAbs for use in the methods herein can be accomplished by labeling T-cells intended for infusion with anti-CD3/anti-CD28 mAb, such as antibodies of the IgG1 subclass, and subsequently mixing the labeled cells with autologous mononuclear cells, generally enriched in granulocytes and macrophages. Fc gammã-RI receptors expressed on neutrophils, monocyte/macrophages and eosinophils have a high avidity for the Fc portion of antibodies, especially of the IgG1 or IgG3 subclasses.

The mixed cells can be suspended in infusion medium and immediately infused into a patient. One way to do this is to mix the labeled cells with autologous mononuclear cells during a leukapheresis procedure. In this manner, the cells are not required to be suspended in infusion medium prior to infusion.

Alternatively, the cells can be mixed with anti-CD3/anti-CD28-conjugated colloidal size particles, dextran coated paramagnetic microbeads beads (Miltenyi Biotec, Auburn Calif.; see, U.S. Pat. No. 6,417,011; see EXAMPLES, below). Such micro-particles remain in suspension since they are colloidal in size. In addition, following binding to CD4 T cells are internalized or shed, as a result the activation signal through CD3 and CD28, is transient and not continuous, and the need to debead the product prior to infusion in patients is eliminated.

The reactivated product is then infused into the patient, which is generally the same as to donor of the original source material.

D. Administration of the gene therapy products The therapeutic methods produce compositions containing clinically relevant (at least 10⁹, typically at least 10¹⁰ cells or more, generally in a volume of a liter, 500 mls, 200 mls, 100 mls or less) populations of polypclonal memory Th1 cells for infusion for treatment of the diseases or conditions characterized by suppression of the cellular immune response, by over-expression of the humoral immune response, excess Th2 activity or a lack or decreased Th1 activity. The methods exemplified herein do not rely or use any agents for expansion or differentiation that must be present after expansion to maintain cell viability or activity.

The compositions contain highly (greater than 70%, 80%, 90% or more of the cells) pure populations of polyclonal memory Th1 cells. Such compositions are used therapeutically for treatment of the diseases, such as cancer, infectious diseases, allergic diseases and other diseases or conditions characterized by suppression of the cellular immune response, by over-expression of the humoral immune response, excess Th2 activity or a lack or decreased Th1 activity.

Administration

The compositions of cell are administered at the satellite facility by any suitable means, including, but not limited to, intravenously, parenterally, or locally. The particular mode selected will depend upon the particular treatment and trafficking of the cells. Typically, about 10¹⁰-10¹¹ cells can be administered in a volume of a 50 ml to 1 liter, 50 ml to 250 ml, 50 ml to 150, and typically 100 ml. The volume will depend upon the disorder treated and the route of administration. The cells can be administered in a single dose or in several doses over selected time intervals in order to titrate the dose.

The cells produced by the methods provided herein can be co-infused with an antigen or the antigen and cells can be administered separately, sequentially or intermittently.

The following examples are included for illustrative purposes only and are not intended to limit the scope of the invention.

### EXAMPLE 1

### Materials and Methods

A. Isolation of human Iymphocytes.

Samples of buffy coats or leukapheresis products from normal donors and EDTA-preserved blood samples from advanced cancer subjects with a variety of indications and prior treatments were used. Human peripheral blood lymphocytes (PBMC) were isolated using a density gradient centrifugation procedure.

B. Characterization of PBMC samples

Purified PBMC samples were characterized by immuno-phenotyping using flow cytometry. Briefly, cells were incubated with fluorochrome-labeled antibodies in the dark for 30 min., washed of excess antibodies and analyzed on FACSCalibur flow cytometer (BD Biosciences). Results of the analysis were expressed as percentages of total lymphocytes, monocytes, granulocytes, and also subsets of lymphocytes: B-cells, cytotoxic T lymphocytes, CD4 positive T-helpers, and NK cells. The subset of CD4 positive T cells was analyzed for the ratio between naïve CD45RA positive cells and CD45RA negative memory cells.

C. Cytokine profiling

To determine the ability of freshly purified CD4 positive cells to express IFN-y and IL-4 an intra-cellular cytokine (ICC) staining procedure using an Internal Cellular Cytokine (ICC) kit (BioErgonomics, St. Paul, Minn. )was performed. According to the manufacturer's recommendation, PBMC were stimulated for 20 h in T-cell activation medium, stained first by surface anti-CD4 antibodies, fixed, permeated and then stained with intracellular anti-IFN-y and anti-IL-4 antibodies. Samples were analyzed by flow cytometry and results were presented as percentages of IFN-y and IL-4 expressing cells in CD4 positive T cells subset.

D. Isolation of T-cell subpopulations

Isolation of specific T-cell subpopulations was performed using two different techniques: sort by flow cytometry on FACSCalibur and sort by combination of positive and negative immunomagnetic selection on AutoMacs (Miltenyi, Germany). To obtain cell samples with high purity, sort by flow cytometry was done. Briefly 4×10⁷ of PBMC were stained with anti-CD4 antibodies alone or in combination with anti-CD45RO antibodies, labeled with the corresponding fluorochrome. Subsets of CD4-positive, CD4-positive/CD45RO-negative and CD4-positive/CD45RO-positive cells were collected by sorting and used for expansion experiments. To obtain better yields with 5-10% lower purities, separation for further applications used immunomagnetic selection.

According to the manufacturer's recommendation, up to 2×10⁸ cells were incubated with anti-CD4 antibodies conjugated directly to magnetic microbeads and separated on magnetic columns. If needed, the second round of selection was performed using mouse anti-CD45RO antibodies in complex with goat anti-mouse antibodies conjugated to microbeads.

E. Activation of cells

Sorted cells were plated into cell culture plates at starting concentrations of 1×10⁵ to 3×10⁵ cells/ml using ex vivo serum free cell culture medium (X-VIVO-15 from BioWhittaker) without supplementation. The cells were cultured for 12 days and were repeatedly activated using a combination of CD3/CD28 antibodies conjugated to magnetic beads (T-cell Expander, Dynal) every 3 days, starting from the day of sort.

Initial cell activation was performed using 3:1 ratio between magnetic beads and sorted cells. For re-stimulation, an amount of beads equal to the amount of cells in the culture determined by hand cell count was used. On day 13, 14 or 15 expanded cell cultures were harvested. The cells were counted cells (manual hand count) and the final product was characterized.

F. Phenotyping

For characterization of the final product, the phenotypes of harvested cells were determined, their ability to express IFN-y and IL-4 by intra-cellular cytokine staining (ICC) and their production of IFN-y, IL-2 and IL-4 (determined by ELISA in the cell culture supernatants of expanded cells before harvesting) were analyzed. Immunophenotyping and ICC experiments were performed as described above. ELISA assays were performed using ELISA kits (R&D, Minneapolis, Minn.) for IFN-y, IL-2, IL-4, IL-10, IL-13, TNF-alpha according to manufacturer's recommendations.

G. Preparation of colloidal size microbeads

Paramagnetic colloidal size beads can be purchased from Miltenyi Biotec (Auburn, Calif.; see, also U.S. Pat. No. 6,417,01 1). As described in U.S. Pat. No. 6,41 7,011, dextran coated paramagnetic colloidal size particles are prepared by mixing 10 g dextran T40 (Pharmacia, Uppsala Sweden), 1.5 g ferric chloride hexahydrate and 0.64 g ferrous chloride tetrahydrate in 20 ml water and heating to 40° C. The solution is stirred and 20 ml 4 M NaOH is added dropwise with continued stirring. The resulting particle suspension is neutralized with acetic acid, centrifuged for 10 min at 2,000 x g, and filtered through a 0.22 μm pore-size filter (Millex GV) to remove aggregates. Unbound dextran is removed by washing in a high gradient magnetic field by washing in columns of steel wool in a high gradient magnetic separation (HGMS) device at a strength of 0.6 Telsa. The particles are washed through the column. These particles can be further derivatized.

### EXAMPLE 2

CD4+ cells were purified from a normal donor. The cells in Group 1 were stimulated with anti-CD3/anti-CD28 only once. The cells in Group 2 were stimulated every 3 days. Both groups were cultured for 14 days.

| | Group 1 | Group 2 |
|---|---|---|
| CD4 | 99.47 | 97.92 |
| CD45RA | 10.29% | 18.23% |
| CD45RO | 16.58% | 81.47% |
| CD62L | 46.97% | 1.92% |
| CD25 | 18.07% | 97.10% |
| CD44 | 99.52% | 99.08% |
| Internal IFN+ | 23.35% | 71.68% |
| Internal IL-4+ | 6.14% | 4.08% |
| IFN ELISA | 1651 pg/ml | 6870 pg/ml |
| IL-4 ELISA | 52 pg/ml | <26.1 pg/ml |

These data indicate that the restimulation process results in an enhanced population of activated (CD25+), memory (CD45RO+) Th1 cells compared to single stimulation methods.

### EXAMPLE 3

T-cells and T-cell subsets were purified from three different cancer subject PBMC by FACS. The blood was purified into four groups: (1) CD3+; (2) CD4+; (3) CD4+, CD45RO− and (4) CD4+, CD45RO+. The cells were stimulated every 3 days with immobilized anti-CD3/anti-CD28 mAb. The resulting cells were analyzed after 14 days of culture to assess their phenotypes.

| | | | CD4+ | CD4+ |
|---|---|---|---|---|
| | CD3+ | CD4+ | CD45RO− | CD45RO+ |
| Subject 1 | | | | |
| CD4+ | 70.94% | 97.76% | 99.52% | 99.01% |
| CD8+ | 20.55% | 0.45% | 0.14% | 1.72% |
| CD45RA+ | 0.89% | 4.01% | 2.95% | 1.62% |
| CD45RO+ | 75.43% | 87.68% | 93.97% | 96.80% |
| CD62L+ | 2.49% | 1.87% | 9.72% | 13.75% |
| CD25+ | 78.98% | 96.02% | 92.97% | 96.08% |
| CD44+ | 79.47% | 99.20% | 99.78% | 99.42% |
| Internal IFN+ | 64.87% | 79.30% | 70.05% | 46.62% |
| Internal IL-4+ | 41.17% | 13.94% | 11.46% | 4.82% |
| IFN ELISA | 1612 pg/ml | 1092 pg/ml | 4332 pg/ml | 2664 pg/ml |
| IL-4 ELISA | <26 pg/ml | <26 pg/ml | <26 pg/ml | <26 pg/ml |
| IL-13 ELISA | 2810 pg/ml | 2227 pg/ml | 986 pg/ml | 703 pg/ml |
| TNF-α ELISA | 8055 pg/ml | 9000 pg/ml | 384 pg/ml | 359 pg/ml |
| IL-10 ELISA | 0 pg/ml | 0 pg/ml | 150 pg/ml | 128 pg/ml |
| Subject 2 | | | | |
| CD4+ | 70.15% | 98.35% | 97.51% | 96.09% |
| CD8+ | 23.53% | 0.42% | 0.19% | 3.65% |
| CD45RA+ | 0.93% | N.D. | 2.02% | 0.15% |
| CD45RO+ | 72.03% | N.D. | 96.47% | 94.06% |
| CD62L+ | 5.18% | N.D. | 20.89% | 13.22% |
| CD25+ | 67.37% | N.D. | 95.22% | 93.85% |
| CD44+ | 68.05% | N.D. | 96.24% | 95.74% |
| Internal IFN+ | 59.62% | 86.09% | 95.71% | 54.78% |
| internal IL-4+ | 5.96% | 11.68% | 9.41% | 3.41% |
| IFN ELISA | 20,868 pg/ml | 25,514 pg/ml | 13,100 pg/ml | 1928 pg/ml |
| IL-4 ELISA | <26 pg/ml | <26 pg/ml | <26 pg/ml | <26 pg/ml |
| IL-13 ELISA | 325 pg/ml | 258 pg/ml | 978 pg/ml | 429 pg/ml |
| TNF-α ELISA | 1427 pg/ml | 1025 pg/ml | 2318 pg/ml | 2318 pg/ml |
| IL-10 ELISA | 380 pg/ml | 800 pg/ml | 320 pg/ml | 1000 pg/ml |
| Subject 3 | | | | |
| CD4+ | N.D. | 98.56% | 97.56% | 98.78% |
| CD8+ | N.D. | 0.07% | 1.75% | 0.16% |
| CD45RA+ | N.D. | 5.17% | 6.27% | 10.40% |
| CD45RO+ | N.D. | 96.60% | 97.36% | 96.00% |
| CD62L | N.D. | 1.30% | 5.55% | 5.21% |
| CD25+ | N.D. | 96.67% | 94.55% | 95.50% |
| CD44+ | N.D. | 99.67% | 97.60% | 99.52% |
| Internal IFN+ | N.D. | 86.63% | 73.45% | 82.03% |
| Internal IL-4+ | N.D. | 2.56% | 4.95% | 3.78% |
| IFN ELISA | N.D. | 4138 pg/ml | 2998 pg/ml | 2798 pg/ml |
| IL-4 ELISA | N.D. | <26 pg/ml | <26 pg/ml | <26 pg/ml |
| IL-13 ELISA | N.D. | 4034 pg/ml | 1746 pg/ml | 679 pg/ml |
| TNF-alpha | N.D. | 2287 pg/ml | 543 pg/ml | 846 pg/ml |
| ELISA | | | | |
| IL-10 ELISA | N.D. | 120 pg/ml | 380 pg/ml | 115 pg/ml |

These data indicate that methods herein generate enhanced populations of activated Th1 memory cells from subject blood with or without purification of T-cell subsets.

### EXAMPLE 4

The following example demonstrates that the method provided consistently produce populations of highly pure, activated, polyclonal memory Th1 cells from a subject blood sample in the absence of any exogenous growth or differentiation factors,

Sixty-four blood samples were obtained. Of these 31 were from patients with metastatic cancer and 33 were from normal donors. The samples included 5 from patients with metastatic breast cancer, 4 from patients with NSCLC (lung cancer), 4 from patients with melanoma, 3 from patients with colon cancer, 2 from patients with prostate cancer, 2 from patients with non-Hodgkin's lymphoma, 2 from patients with pancreatic cancer, 2 from patients with liver cancer, 1 from a patient with cervical cancer, 1 from a patient with ovarian cancer, 1 from a patient with renal cell carcinoma, 1 from a patient with esophageal cancer, 1 from a patient with head and neck cancer, 1 from a patient with brain cancer and 1 from a patient with stomach cancer.

Mononuclear cells were isolated from 50 ml peripheral blood samples by density gradient centrifugation. CD4+ cells were purified by positive selection with biotyinylated anti-CD4 mAB and anti-biotin microbeads (Miltenyi) in a magnetic field. The CD4+ cells were incubated for 14 days in serum-free medium with no supplements. Cells were stimulated every 3 days with anti-CD3/anti-CD28 monoclonal antibody conjugated 4.5 micron paramagnetic beads. The resulting populations of cells were as follows:

| Description of | | |
|---|---|---|
| phenotype of cells | Cancer donors (n = 31) | Normal donors (n = 33) |
| % CD4+ | 97.70 ± 2.97 | 97.14 ± 5.35 |
| % CD4+/CD45RO+ | 94.17 ± 10.68 | 88.70 ± 21.88 |
| % CD4+/CD62low | 88.99 ± 13.16 | 86.83 ± 14.69 |
| % CD4+/CD25+ | 95.72 ± 6.41 | 93.06 ± 16.54 |
| % CD4+/CD44+ | 99.36 ± 0.80 | 98.97 ± 2.30 |
| % CD4+/CD40L+ | 40.90 ± 14.26 | 61.38 ± 23.32 |
| ICC IFN-γ | 74.77 ± 14.14 | 61.12 ± 26.32 |
| ICC IL-4 | 13.46 ± 15.53 | 13.79 ± 13.27 |

*ICC indicates internal cytokine staining;
**Percentage of cells staining positive for the phenotypic marker by standard flow cytometry methods, expressed as the mean ± standard error.

These results show that the methods produced population of cells that do not vary significantly from patient and between cancer patients an normal donors as is observed using other methods. This indicates that the method reproducibly produces a consistent end product that will not vary from batch-to-batch.

### EXAMPLE 5

The following data show a time course of the production of IFN-y, IL-4 and IL-2 (ELISA; pg/ml) as a function of days in culture for various samples from three different cancer subjects restrimulating the cells every three days. Th1 differentiation correlates with IFN-y production for each subject.

| | IFN-γ | IL-4 | IL-2 |
|---|---|---|---|
| Subject 1 | | | |
| day 1 | 99.1 | 26.1 | 1029 |
| day 2 | 87.3 | 26.1 | 1651.7 |
| day 3 | 120.3 | 67.5 | 6151.87 |
| day 4 | 174.6 | 58.3 | 1116.8 |
| day 5 | 164.1 | 28.5 | 186.1 |
| day 6 | 187.2 | 26 | 101.2 |
| day 7 | 761.4 | 27.2 | 319 |
| day 8 | 1672.3 | 25 | 50 |
| day 9 | 1521.2 | 25 | 50 |
| day 10 | 2500 | 25 | 50 |
| day 12 | 2500 | 25 | 50 |
| harvest | 1003 | 25 | 150 |
| Subject 2 | | | |
| day 1 | 45 | 15 | 366 |
| day 2 | 60 | 15 | 3000 |
| day 3 | 900 | 78 | 7500 |
| day 4 | 3900 | 108 | 7500 |
| day 5 | 4500 | 15 | 5500 |
| day 6 | 6300 | 15 | 200 |
| day 7 | 6900 | 15 | 3210 |
| day 8 | 6900 | 15 | 783 |
| day 9 | 6900 | 15 | 170 |
| day 10 | 7200 | 15 | 636 |
| day 11 | 7200 | 15 | 1300 |
| day 12 | 7200 | 15 | 1800 |
| harvest | 7200 | 00 | 1585 |
| Subject 3 | | | |
| day 4 | 120.1 | 92.6 | 152.1 |
| day 5 | 154.6 | 129.9 | 159.1 |
| day 6 | 193.8 | 76.9 | 150 |
| day 7 | 290.8 | 28.14 | 150 |
| day 9 | 910.9 | 25 | 150 |
| day 12 | 7387 | 25 | 150 |
| harvest | 7000 | 25 | 150 |

These data also demonstrate that IFN-y, and thus, Th1 differentiation, peaks between about day 9 to day 12.

### EXAMPLE 6

### Restimulation of rested T-cells

Preparation of T-cells

Pure Th1 cells were prepared by the frequent and repeated activation method exemplified and described in EXAMPLES 1-5 and described in copending U.S. application Ser. Nos. 09/957,194 and 10/071,016. Briefly, CD4+ cells were purified by positive selection from patients with advanced cancer. The cells were cultured in X VIVO-15 culture medium supplemented with glutamine. On day 10, the cells were incubated with anti-CD3/anti-CD28 conjugated immunomagnetic beads at a 3:1 bead:cell ratio. Every 3 days the cells were restimulated at a 1:1 ratio. On day 14, two days after last stimulation, the cells were harvested and separated from the beads.

Restimulation

The day 14 harvested cells were washed and resuspended in fresh medium. The cells were divided into two groups and each group of cells was incubated for 120 hours. The first group (no restimulation) was cultured without any activation. The second group (restimulation) was restimulated after 72 h (120 h after last stimulation). Samples were taken every 24 h and analyzed by ELISA for cytokine production. For restimulation at 72 h, cells were removed, labeled with anti-CD3 and anti-CD28 mAb and mixed at a 1:3 ratio with freshly collected autologous PBMC.

| | 24 h | 48 h | 72 h | 96 h | 120 h |
|---|---|---|---|---|---|
| IFN-gamma | | | | | |
| pg/ml/1 million | | | | | |
| No restimulation | 3850 | 2240 | 1050 | 680 | 180 |
| restimulation | | | 98,800 | 42,500 | 14,600 |
| IL-2 | | | | | |
| pg/ml/1 million | | | | | |
| No restimulation | 120 | 60 | <50 | <50 | <50 |
| restimulation | | | 2800 | 580 | <50 |
| IL-4 | | | | | |
| pg/ml/1 million | | | | | |
| No restimulation | <26 | <26 | <26 | <26 | <26 |
| restimulation | | | 50 | 35 | <26 |

These data demonstrate that restimulation of primed cells that have rested 120 h after removal from an activation stimulus results in significant increases in cytokine production.

### EXAMPLE 7

### Summary of cytokine production data of Day 14 harvested cells that were last stimulated on Day 9

The cells were removed from the beads on Day 14 and cultured for 24 h. One group was labeled with anti-CD3/anti-CD28 and mixed with autogous PBMC at a 1:2 ratio. A second group was stimulated with anti-CD3/anti-CD28 conjugated beads and a third group was not restimulated. Cytokine production at 4 h and 24 h was analyzed by ELISA.

| | 4 hr | 4 hr | 4 hr | 24 hr | 24 hr | 24 hr |
|---|---|---|---|---|---|---|
| | *IFN-γ | IL-4 | **TNF-α | *IFN-γ | IL-4 | **TNF-α |
| | (pg/ml) | (pg/ml) | (pg/ml) | (pg/ml) | (pg/ml) | (pg/ml) |
| Day 14 | 526.17 ± | 6.3 ± 0.00 | 103.11 ± 35.68 | 4625 ± | 6.47 ± 0.29 | 252.10 ± |
| harvested cells | 702.26 | | | 877.46 | | 77.70 |
| only (HRV) | | | | | | |
| 1:2 | 2502.45 ± | 7.4 ± 2.34 | 408.45 ± 15.10 | 21982.86 ± | 20.44 ± 18.32 | 2665.20 ± |
| HRV:PBMC | 3070.93 | | | 21013.53 | | 432.31 |
| w/sol 3/28 | | | | | | |
| HRV Cell + | 3338.45 ± | 6.79 ± 0.98 | 517.24 ± 41.22 | 15920.95 ± | 9/38 ± 2.24 | 3084.00 ± |
| CD3/CD28 | 3581.14 | | | 6440.41 | | 1756.93 |
| Beads | | | | | | |

*The concentrations of IFN-γ were normalized to 1.0 × 10⁶ HARVESTED cells/ml.
**The concentrations of TNF-α were normalized to 1.0 × 10⁶ TOTAL cells/ml.
The sample size (n) was 8 and included 6 normal donors and 2 cancer donors.

These data demonstrate that restimulation of primed, resting cells prior to infusion results in cells with significantly enhanced cytokine production. The amount of cytokine production is so high as to raise concerns about potential toxicity. This experiment demonstrates that only low amounts of cytokines are produced within the first 4 hours after re-stimulation and that the cytokine production peaks around 24 hours post re-stimulation. This indicates that these re-stimulated cells should be infused within 4 hours of re-stimulation. If the cells have an activated memory phenotype (CD45RO +, CD25 +, CD62L^{Lo}), which have an activated memory phenotype (CD45RO +, CD25+, CD62L^{Lo}). Cells with such phenotype are expected to extravasate and enter areas of inflammation. By administering these cells by four hours, they will enter the areas prior to peak cytokine production. Local cytokine production is known to be less toxic than systemic cytokine production.

### EXAMPLE 8

When looking cytokines produced as a function of the ex-vivo immunotherapy process (i.e. from initial culture to harvest to re-infusion) it was found that in culture the cells general increase in cytokine productions. The cells are then harvested from the culture environment, and formulated (i.e., washed and put into infusion medium). Typically there is delay from formulation to infusion, such as for shipping. The problem, as shown herein, is that by the time the patient and cells are ready for infusion, there is no cytokine production. At that point, cell viability is also significantly decreased. This example presents the results of a study to determine how to keep the cells viable and producing cytokines.

Viability Study

Purified CD4+ cells were activated with anti-CD3/anti-CD28 conjugated beads every 3 days for 9 days. On day 12, the cells were harvested, washed and resuspended at 1×10⁸ cells/ml in various infusion media. These formulated cells were stored for 48 hours at either 4° C., 22° C. or 37° C. The cells from each batch were formulated in saline, 5% dextrose, Plasma-Lyte, Normosol or autologous plasma. Samples were taken at 4 h, 12 h, 24 h and 48 h and analyzed for viability and production of interferon-gamma. Each table presents a different formulation of infusion medium, the numbers are the percent viable cells ± standard error. The data represent the results of 6 different patients.

| | | | |
|---|---|---|---|
| 4 h | 12 h | 24 h | 48 h |

Saline

| | | | | |
|---|---|---|---|---|
| 37° C. | 72 ± 14 | 58 ± 20 | 42 ± 18 | 22 ± 12 |
| 22° C. | 93 ± 13 | 82 ± 17 | 48 ± 15 | 26 ± 14 |
| 4° C. | 92 ± 6 | 80 ± 12 | 52 ± 18 | 48 ± 20 |

5% Dextrose

| | | | | |
|---|---|---|---|---|
| 37° C. | 68 ± 12 | 62 ± 14 | 50 ± 20 | 35 ± 25 |
| 22° C. | 94 ± 6 | 90 ± 10 | 82 ± 6 | 20 ± 20 |
| 4° C. | 89 ± 9 | 78 ± 20 | 68 ± 18 | 50 ± 12 |

Plasma-Lyte

| | | | | |
|---|---|---|---|---|
| 37° C. | 92 ± 8 | 80 ± 12 | 75 ± 16 | 25 ± 25 |
| 22° C. | 96 ± 4 | 90 ± 8 | 83 ± 10 | 55 ± 18 |
| 4° C. | 94 ± 8 | 92 ± 10 | 84 ± 12 | 62 ± 15 |

Autologous Plasma

| | | | | |
|---|---|---|---|---|
| 37° C. | 98 ± 2 | 97 ± 2 | 93 ± 6 | 85 ± 8 |
| 22° C. | 99 ± 1 | 97 ± 2 | 96 ± 3 | 89 ± 6 |
| 4° C. | 93 ± 4 | 85 ± 8 | 80 ± 10 | 78 ± 12 |

Normosol

| | | | | |
|---|---|---|---|---|
| 37° C. | 93 ± 7 | 82 ± 14 | 70 ± 12 | 28 ± 16 |
| 22° C. | 93 ± 7 | 85 ± 6 | 78 ± 16 | 58 ± 16 |
| 4° C. | 90 ± 8 | 80 ± 6 | 80 ± 12 | 48 ± 20 |

These results demonstrate that cells formulated in infusion medium exhibit a significant decrease in viability notable within the first 12 h after formulation. Increased temperature results in more rapid loss of viability and decreased temperature slows the loss of viability. Formulation in autologous plasma was capable of maintaining cell viability. In addition, in the cells in group formulated in autologous plasma was the cytokine production maintained. Representative data from one culture is shown below:

IFN-Gamma Cytokine Production at 22° C. (pg/ml)

| | 4 h | 12 h | 24 h | 48 h |
|---|---|---|---|---|
| Saline | ND | ND | ND | ND |
| 5% Dextrose | ND* | ND | ND | ND |
| Plasma-Lyle | 240 | 80 | ND | ND |
| Normosol | 280 | 120 | ND | ND |
| Autologous Plasma | 9600 | 6200 | 4800 | 2200 |

*ND = not detectable

As shown in the Example, below, reactivation in infusion medium containing antibody-conjugated colloidal size particles also maintains viabilty, since the particles do not have to be removed prior to infusion.

### EXAMPLE 9

### Preparation of anti-CD3 and anti-CD28 monoclonal antibody colloidal paramagnetic beads

Human anti-CD3 and anti-CD28 mouse Monoclonal antibodies are immobilized on Miltenyi Goat-Anti-Mouse (GAM) micro-beads for Th1 cell expansion. The beads are used for activation of primed CD4+ T cells (CD4+ T cells activated using Human anti-CD3 and anti-CD28 immobilized on Dynal beads). Advantages of using these beads include, for example: 1) The Miltenyi beads are micro particles that remain in colloidal suspension, as a result these beads do not settle at the bottom of the flask in bioreactor; 2) Miltenyi micro-particles following binding to CD4 T cells will be internalized or shed, as a result the activation signal through CD3 and CD28 will be transient and not continuous; and 3) the need to debead the product prior to infusion in patients is eliminated.

A. Materials:

Goat anti-Mouse IgG Miltenyi Microbeads

Dulbecco's Phosphate Buffered Saline (dPBS)

General Buffer (dPBS with 1% HSA)

OKT3 human anti-CD3 monoclonal Antibody, 1 mg/ml (Ortho)

CD28 ASR, human anti-CD28 Bulk monoclonal Antibody, 1 mg/ml (BD)

MS or LS column for MiniMACS or OctoMACS (Miltenyi order # 130-042-201 or 130-042-401)

MiniMACS (Miltenyi order # 130 042 302)or MidiMACS unit (Miltenyi order #130 042 102)

Sample CD3/CD28 Antibody Solution for Quality Control

Sample CD3/CD28 Expansion Beads for Quality Control

Miltenyi CD3/CD28 T-Cell Expansion Beads

B. Preparation

To prepare the beads, human anti-CD3 and anti-CD28 were mixed at ratio of 1:1 and added to the solution of beads. The mixture of beads and antibodies was incubated room temperature. The beads were washed on a Miltenyi MS column 10 times to remove unbound antibodies and eluted from the column using X-vivo15.

To prepare the volume of beads to be conjugated is selected. Each 2 mL of GAM Miltenyi beads results in about 2 mL of anti-CD3/anti-CD28 beads. Twenty μL of anti-CD3/anti-CD28 beads were required to stimulate ≦10⁷ total cells. The colloidal solution of GAM Miltenyi beads was gently vortexed to re-suspend the beads, which were then transferred to a 12×75 polypropylene tube for coupling the antibodies.

To prepare a CD3/CD28 antibody solution, CD3 and CD28 antibodies were mixed together in equal amounts to produce a homogeneous solution. For each 500 μl of GAM beads 100 μg each of anti-CD3 and anti-CD28 antibody solution was used. The solution was produced by mixing equal amounts of anti-CD3 and anti-CD28 antibodies.

For conjugation of the antibodies to the beads, 200 μl of anti-CD3/anti-CD28 solution was added for every 500 μl of GAM Miltenyi beads and the resulting mixture is gently vortexed. The antibody-bead solution tube was placed on spindle rotors for 60 minutes at room temperature.

To remove the unbound antibody, an MS column was assembled in the magnetic field of an OctoMACS separator (Miltenyi Magnet). A collection tube was placed under the column. 500 μl of degassed PBS buffer was placed on top of the column and run through to pre-equilibrate. The bead-antibody solution was loaded onto the pre-equilibrated column. Antibody-bead solution was run through, and unbound antibody in the effluent was collected. The column was washed with 10×500 μL General Buffer (dPBS with 1% HSA) and total effluent collected as negative fraction (contains unbound antibody). 500 μl of X-Vivo 15 was applied to the column and the beads pushed out and stored in a sterile 50 mL conical centrifuge tube at 4° C.

### EXAMPLE 10

### Th1 Cell Preparation using antibodies immobilized on nanobeads for re-stimulation

A. Preparation of the Th1 cells

As in the above Examples, leukocytes (˜5000 ml) were obtained from Donor/Patients by leukapheresis. The leukapheresis product was further purified using magnetic separation techniques, described above, to isolate that CD4 cell fraction (>80% pure). The CD4 cell fraction and anti-CD3/anti-CD28 immobilized Dynal beads were incubated together for 3 days. Briefly, approximately 25×10⁶purified CD4+ cells were placed in a sterile 12×75 culture tube with cap. The cells were centrifuged and the supernatant discarded. The cells were resuspended in 2.5 mL X-VIVO 15 Medium (10×10⁶ cells per mL).

1875 μL of CD3/CD28 coated sheep anti mouse lGg (SAM) Dynabeads (4×10⁷ beads/mL at a 3:1 ratio of beads to cells) were dispensed into a 50 mL conical centrifuge tube, which was placed into the MPC Magnet and rocked gently 5 times to expose all of the liquid to the magnet. At the end of 5 minutes, with the tube on the magnet, the supernatant X-Vivo 15 medium was removed. The tube was then removed from the magnetic field. The beads were gently disturbed by tapping the tube.

The purified CD4+ cells gently mixed with the bead pellet by tapping. The tube was placed on ice for 20 minutes and vortexed gently every 5 minutes during this incubation. 22.5 mL of pre-warmed X-Vivo 15 was added to the 2.5 mL bead/cell mixture for a final concentration of 1×10⁶ cells/mL. These cells were inoculated into a culture bag (LifeCell). The bag was placed in a 37° C. incubator at 5% CO₂ and 100% humidity. The Dynal Beads were removed by magnetic separation resulting in a cell culture mixture.

B. Restimulation and expansion of Th1 Cells in Bags

The cell culture mixture is re-stimulated with anti-CD3/anti-CD28 immobilized on GAM Miltenyi microbeads (Miltenyi Biotec, Auburn Calif.), prepared as described in the above EXAMPLE 9. At day 3 (72 hours post initiation, the contents of the bag were gently but thoroughly mixed, and then transferred to a 50 mL conical centrifuged tube, which was placed into the MPC Magnet for 5 minutes. The supernatant was removed and into a fresh 50 mL conical centrifuge tube.

About 5.0 mL of the well-mixed cell suspension was transferred into each of 2 tubes for analyses. The tube containing the bulk of the cell suspension was centrifuged at 1200 rpm for 5 minutes, and the supernatant was transferred into another sterile 50 mL conical centrifuge tube, centrifuged and resuspended in conditioned medium at a cell density of 100×10⁶ cells/mL (WBC count from Sysmex×volume of medium×% viability)/100=mL of conditioned medium to add) and placed on ice.

20 μL of anti-CD3/anti-CD28-GAM-Miltenyi microbeads per 10×10⁶ cells was added, mixed well, incubated on ice for 20 minutes, vortexing gently every 5 minutes during the incubation.

When incubation is complete the density is adjusted by addition of a 25:75 (v/v) mixture of conditioned medium and fresh X-Vivo 15 to 1×10⁶ cells/mL. These cells are inoculated into a new culture bag and incubated. Each day of the incubation, a portion of the working supernate is exchanged for fresh medium to replenish nutrients and remove waste products.

On day 6 and day 9 of the culture, the cell culture mixture is re-stimulated with anti-CD3/Anti-CD28 immobilized on GAM Miltenyi Micro Beads. 20 μL of anti-CD3/anti-CD28-GAM-Miltenyi microbeads was added per 10×10⁶ cells that had been resuspended at a density of 100 ×10⁶/mL. The tube containing the cell/bead suspension was placed into an ice bath for twenty minutes and mixed gently every five minutes during the cold incubation, the cells bead mixture is transferred to a culture bag, which was placed in the incubator. On day 13, the cells were harvested.

C. Results

The resulting cells had the following properties, which indicate that they are polyclonal Th1 cells:

A. Purity ˜99% CD4+ and CD3+.

B. Viability>90%

C. Produce a large amount of INF-gamma cytokine (up to about 10 ng per million cells)

D. Do not produce detectible IL-4

E. Do not produce detectible TGF-beta

F. Do not produce detectible IL-10.

G. Do not have detectible CTLA-4 on cell surface.

H. At gene expression level these cells exhibit:

1. detectible expression of INF-gamma, IL-2, IL-1 5, IL-18, TNF-alpha, TNF-beta.

2. undetectable IL-4, IL-10, IL-5, IL-12P35, IL-12P40, IL-1beta, IL-150 alpha, IL-6 expression.

### EXAMPLE 11

### Reactivation of cells using CD3/CD28 antibodies conjugated to colloidal size paramagnetic beads

Resting Th1 cells harvested cells produced by any method can be used. In this example, the Th1 cells were produced by the method in EXAMPLES 2-5. The cells were washed to remove all supernatant liquid, such as medium from the cell culture), and placed in fresh medium and re-stimulated with anti-CD3/anti-CD28 immobilized on Miltenyi micro beads as in EXAMPLE 9.

After the initial incubation, the cells were re-suspended in new clean medium and allowed to incubate 24 hours. The resulting cell culture mixture has all of the characteristics associated with cells when originally harvested. In was found that these cells exhibit augmented INF-gamma production following stimulation (up to 2.5 ng per million cells in 24 hours).

Since modifications will be apparent to those of skill in this art, it is intended that this invention be limited only by the scope of the appended claims.

1. A therapy method, comprising:
a) establishing a central processing facility and a plurality of satellite facilities for conducting somatic cell or gene therapy;
b) collecting source biological material at one of the satellite facilities from a first subject;
c) transporting the source material from the first subject and delivering the source material to the central processing facility;
d) processing the source material from the first subject at the central processing facility to produce a therapy product; and
e) transporting the therapy product back to the satellite facility for administration to a subject, wherein all steps are performed, such that the manufacturer has vein-to-vein control over the processes, facilities and products, and source material is tracked from collection through processing to infusion of the product derived from the source material.

2. The method of claim 1, wherein all steps are performed under the control of one manufacturer, which controls personnel, facilities, equipment, documentation and procedures used at the collection and administration of somatic cell and gene therapy products.

3. The method of claim 1, wherein all steps are performed under a single government license.

4. The method of claim 1, wherein each of the plurality of satellite facilities are located in distinctly separate subject locations.

5. The method of claim 1, further comprising establishing at least one more additional central processing facility.

6. The method of claim 1 wherein the therapy product is an autologous product.

7. The method of claim 1, wherein the satellite facilities are connected to the central processing facility through a global computer network in which data specific to the first subject is collected, transmitted and stored for use for processing the source material and for administering the therapy product back to the first subject.

8. The method of claim 4, further comprising:
labeling source material collected for shipment to the central processing facility; and
transporting the therapy product made from the source material back to the satellite facility for administration to the first subject with an identifying symbology.

9. The method of claim 8, wherein the symbology is an optically readable label.

10. The method of claim 8, wherein the label identifies the subject from whom the material was obtained.

11. The method of claim 7, further comprising:
creating a digital photograph of the first subject to ensure positive subject identification and association with the source material collected and for positive identification and association for administering the resulting therapy product to the same first subject.

12. The method of claim 1, wherein:
source material is collected from a plurality of subject;
the first subject is one of a plurality of subjects at each of the satellite facilities;
the source material is transported to the central processing facility to produce therapy products specific to each of the plurality of subjects derived from source material obtained from such subjects; and
transporting each of the therapy products back to the satellite facility from which the source material was obtained and administering the therapy product to the same subject from which the source material was obtained.

13. The method of claim 8, wherein the satellite facilities are connected to the central processing facility through a global computer network in which data for each subject from which source material is collected is entered, associated with the source material, and stored for use for processing the source material, and then used for transporting back the therapy product produced from the source material for each subject and for administering the therapy product back to the subject from which the original source material that produced the specific therapy product was obtained.

14. The method of claim 1, wherein processing in step d) is effected by a method, comprising:
i) purifying T-cells from the source material; and
ii) activating the T-cells a minimum of 3 times at 2-4 day intervals, whereby a highly pure population of polyclonal Th1 memory cells is produced.

15. The method of claim 14, wherein the T-cells are purified CD4+ cells.

16. The method of claim 14, wherein the CD4+ cells are purified by positive selection

17. The method of claim 16 wherein the CD4+ cells are purged of CD45RO+ cells

18. The method of claim 14, wherein the source material is purged of platelets

19. The method of claim 17, wherein the source material is purged of platelets

20. The method of claim 14, wherein the source material is purged of monocytes.

21. The method of claim 19, wherein the source material is purged of monocytes.

22. The method of claim 14, wherein the activation of T-cells is effected by contacting the cells by contacting with anti-CD3 and anti-CD28 monoclonal antibodies (mAbs).

23. The method of claim 22, wherein the anti-CD3 and anti-CD28 mAbs are immobilized.

24. The method of claim 22, wherein the anti-CD3 and anti-CD28 mAbs are immobilized on particles.

25. The method of claim 24, wherein the particles are initially administered to the purified T-cells at a 3:1 particle:cell ratio and, in subsequent steps, at a 1:1 particle:cell ratio.

26. The method of claim 1, wherein the subject has a disease characterized by either an excess of Th2 cytokine activity or low Th1 cytokine activity.

27. The method of claim 1, wherein the subject has a disease selected from the group consisting of diseases characterized by suppression of the cellular immune response or by over-expression of the humoral immune response.

28. The method of claim 1, wherein the subject has a disease selected from the group consisting of cancer, infectious diseases, autoimmune and allergic diseases.

29. The method of claim 14, wherein the subject has a disease selected from the group consisting of diseases characterized by suppression of the cellular immune response or by over-expression of the humoral immune response.

30. The method of claim 14, wherein the subject has disease selected from the group consisting of cancer, infectious diseases, autoimmune and allergic diseases.

31. The method of claim 1, wherein processing at step d) is effected by a method, comprising:
(i) reducing the number of platelets in the sample;
(ii) purging macrophages from the sample;
(iii) purging the CD45RO + cells from the sample
(iv) purifying by positive selection a population of CD4+, CD45RA+ cells;
(v) activating the CD4+ cells in the absence of exogenous cytokines with immobilized anti-CD3/anti-CD28 mAb; and
(vi) periodically restimulating with immobilized anti-CD3/anti-CD28 mAb.

32. The method of claim 31, wherein, at step (vi), the cells are restimulated every 2-3 days with immobilized anti-CD3/anti-CD28 mAb for a total of 10-14 days.

33. The method of claim 31, wherein, after transporting the cells to the satellite facility, the cells from the resulting cell therapy product containing Th1 memory cells are infused into the subject, thereby altering the Th1/Th2 cell balance of the subject.

34. The method of claim 14, wherein the resulting cell therapy product is transported to a satellite facility and then cells in the product are re-activated cells prior to infusion.

35. The method of claim 14, wherein the harvested cells are frozen, transported to satellite facility, thawed, and then reactivated prior to infusion.

36. The method of claim 34, wherein, the cells are re-activated no more than about 4 hours prior to infusion.

37. The method of claim 35, wherein, the cells are re-activated no more than about 4 hours prior to infusion.

38. The method of claim 34, wherein, prior to re-activating, the cells are rested for about 24 to about 120 hours, wherein resting includes time for transporting form a central processing facility to a satellite facility and or after transporting to a satellite facility.

39. The method of claim 38, wherein the cells are rested for about 72 to about 96 hours before reactivation.

40. The method of claim 38, wherein all or a portion of the resting period occurs after processing and during transporting the therapy product back to a satellite facility.

41. The method of claim 38, wherein all or a portion of the resting period occurs after processing and after transporting the therapy product back to the satellite facility.

42. The method of claim 34, wherein re-activation is effected by
contacting the cells with activating monoclonal antibodies; and then
mixing them with peripheral blood monocytes.

43. The method of claim 34, wherein cells from the resulting cell therapy product are then infused into the patient from whom the source biological material was removed.

44. The method of claim 1, wherein the therapy product comprises purified CD4+ cells.

45. The method of claim 1, wherein the therapy product comprises T-cells suspended in plasma, wherein the plasma is autologous with respect to the T-cells.

46. The method of claim 1, wherein the therapy product comprises T-cells at a density of at least about 10⁷ T-cells per ml.

47. A cell therapy system that for production of a therapy product, comprising:
a plurality of satellite facilities, wherein each satellite facility collects a source material from a subject, administers the therapy product to the subject, or any combination of any of these; and
a central processing facility that is connected to each satellite facility, wherein the central processing facility processes the source material to form the therapy product, wherein:
a single manufacturer controls all satellite and central processing facilities; and
the cell therapy system is effective to collect source material from a subject, transform the source material into the autologous therapy product, and administer the therapy product to a subject.

48. The system of claim 47, wherein each satellite facility and the central processing facility operate under a single government license for conducting somatic cell therapy or gene therapy.

49. The system of claim 47, further comprising computer network connections between and among the central processing facility and each satellite facility.

50. The system of claim 49, wherein the network is a secure global computer network.

51. The system of claim 47, wherein each facility collects the source material, administers the therapy product or any combination of any of these.

52. The system of claim 47, wherein the central processing facility transforms the source material into the autologous therapy product.

53. The system of claim 47, further comprising means for tracking chain-of-custody of source material from collection to infusion.

54. A method of producing an autologous somatic cell therapy product, comprising:
processing a subject at a satellite facility to identify and track the subject;
obtaining source material from the subject;
transforming a source material into the autologous somatic cell therapy product at a central processing facility connected to the satellite facility via a computer network,
wherein all steps are performed, such that the manufacturer has vein-to-vein control over the processes, facilities and products.

55. The method of claim 54, wherein the satellite facility is located at a separate facility from the central processing facility.

56. The method of claim 54, wherein the satellite facility and the central processing facility are administered under a single government license for conducing somatic cell therapy or gene therapy.

57. The method of claim 54, further comprising, transporting a cell therapy product from a central processing facility, wherein the transporting and tracking thereof are controlled by the manufacturer.

58. The method of claim 54, further comprising identifying the subject from whom the source material was obtained, and administering the cells to the subject.

59. The method of claim 1, wherein the subject is a human.

60. The method of claim 1, further comprising:
infusing cells from the resulting cell therapy product into the subject from whom the source biological material was removed.

61. The system of claim 53, wherein the means comprise an optical reading and writing system.

62. The system of claim 61, wherein the optical reading an writing system generates and reads bar codes.

63. The system of claim 47, further comprising documentation and protocols for performing obtaining source material for cell therapy and preparing cell therapy products under a single governmental license.
